(19) United States
(12) Patent Application Publication
(10) Pub. No.: US 2009/0176655 A1
(43) Pub. Date: Jul. 9, 2009

(54) Methylation detection

(75) Inventor: Manel Esteller, Madrid (ES)

Correspondence Address: FOLEY AND LARDNER LLP;SUITE 500, 3000 K STREET NW, WASHINGTON, DC, 20007, US

(73) Assignee: OncoMethylome Sciences SA

(21) Appl. No.: 12/292,289

(22) Filed: Nov. 14, 2008

Related U.S. Application Data
(60) Provisional application No. 60/988,670, filed on Nov. 16, 2007.

(51) Int. Cl. C40B 30/04; C12Q 1/68; C07H 21/04

(52) U.S. Cl. 506/9; 435/6; 536/24.33

(57) ABSTRACT

A method of identifying nucleic acid molecules differentially methylated in a disease comprises steps of incubating fragmented DNA, from a disease cell, with a reagent which specifically binds to methylated DNA to thus concentrate methylated DNA fragments, incubating fragmented DNA, from a disease cell related to the disease cell utilised in step (a) in which DNA methyltransferase expression and/or activity has been inhibited, with a reagent which specifically binds to methylated DNA to thus concentrate methylated DNA fragments and comparing the methylated DNA fragments obtained in steps (a) and (b) to identify nucleic acid molecules differentially methylated in the disease. A method of detecting a predisposition to, or the incidence of, colorectal cancer in a sample comprises detecting an epigenetic change in at least one gene selected from RASGRF2, SCNN1B, HOXD1, PLK2 and BHLHB9 wherein detection of the epigenetic change is indicative of a predisposition to, or the incidence of, colorectal cancer.

### CROSS-REFERENCE TO RELATED APPLICATION

This application claims the benefit of priority from U.S. Provisional Application No. 60/988,670, filed on Nov. 16, 2007. The contents of that application is hereby incorporated by reference in its entirety.

### FIELD OF THE INVENTION

The present invention relates to detection of epigenetic modifications, in particular methylation. More specifically the invention relates to methods of identifying nucleic acid molecules differentially methylated in a disease. The invention also identifies new markers differentially methylated in the disease state.

### BACKGROUND TO THE INVENTION

The inactivation of tumor suppressor genes in human cancer occurs through intragenic mutations, genomic deletions, and also very often by epigenetic silencing associated with the hypermethylation of the CpG islands located in the promoter regions of these genes (1-3). Examples of widely recognized tumor suppressor genes undergoing CpG island promoter hypermethylation in sporadic tumors include the cell cycle inhibitor p16INK4a, the DNA mismatch-repair gene hMLH1, and the breast cancer gene BRCA1 (1-3). Global cytosine methylation patterns in mammals appear to be established by a complex interplay of at least three independently encoded DNA methyltransferases (DNMTs): DNMT1, DNMT3a, and DNMT3b (1-3). The generation of somatic cell knockouts through homologous recombination is a powerful method by which we may clarify the function of any candidate gene in human cancer. Homologous recombination has been used in the colorectal cancer cell line HCT-116 to disrupt DNMT1 or/and DNMT3b (4, 5). Single DNMT knockouts had minor changes in DNA methylation (4, 5) that, in part, might be associated with the presence of recently identified alternative transcripts arising from the DNMT1 gene (6). However, the HCT-116 double knockout cells for DNMT1 and DNMT3b (DKO cells) (5) showed a minimal DNA methyltransferase activity, a 95% reduction in 5-methylcytosine content, demethylation of repeated sequences, loss of imprinting at the IGF2 locus, and abrogation of the methylation-mediated silencing of the tumor suppressor genes p16INK4a and TIMP-3 (5).

Among others, the candidate gene, genomic and pharmacological approaches have all been used in the search for new genes that undergo methylation-associated inactivation in cancer cells (1-3), but the DNMT genetic avenue has not yet been fully explored.

### SUMMARY OF THE INVENTION

The present invention provides a method of identifying nucleic acid molecules differentially methylated in a disease comprising, consisting essentially of or consisting of

(a) incubating fragmented DNA, from a disease cell, with a reagent which specifically binds to methylated DNA to thus concentrate methylated DNA fragments
(b) incubating fragmented DNA, from a disease cell related to the disease cell utilised in step (a) in which DNA methyltransferase expression and/or activity has been inhibited, with a reagent which specifically binds to methylated DNA to thus concentrate methylated DNA fragments
(c) comparing the methylated DNA fragments obtained in steps (a) and (b) to identify nucleic acid molecules differentially methylated in the disease.

In certain embodiments, step (c) comprises, consists essentially of or consists of differentially labelling the methylated DNA fragments obtained in steps (a) and (b) and hybridizing the methylated DNA fragments to a microarray to identify nucleic acid molecules differentially methylated in the disease

In specific embodiments, nucleic acid molecules differentially methylated in the disease are further characterised by determining the presence or absence of a CpG island in the nucleotide sequence. Such methods may further comprise, consist essentially of or consist of determining the methylation status of the CpG island of the nucleic acid molecules in a disease cell to determine whether there is hypermethylation of the CpG island in the disease cell. The methylation status of the CpG island of the nucleic acid molecules from both a disease cell and a disease cell in which DNA methyltransferase expression and/or activity has been inhibited may be determined to identify nucleic acid molecules which are methylated in the disease cell but unmethylated or methylated to a lesser extent in the disease cell in which DNA methyltransferase expression and/or activity has been inhibited.

The methods may, in certain embodiments, further comprise, consist essentially of or consist of determining the methylation status of the CpG island of the nucleic acid molecules which include a CpG island in a non-disease cell wherein a lack of methylation or a lesser degree of methylation in the non-disease cell (as compared to the level of methylation in the disease cell) indicates that the nucleic acid molecule is methylated as an indicator of the disease.

In certain embodiments, the methods further comprise, consist essentially of or consist of determining the effect of methylation on expression of the nucleic acid molecule by comparing gene expression in the disease cell and disease cell in which DNA methyltransferase expression and/or activity has been inhibited. The methods may also involve determining whether use of a demethylating agent can restore expression of the nucleic acid molecule in the disease cell.

The methods of the invention may be utilised to identify candidate tumour suppressor genes.

In a further aspect, the invention provides a method of detecting a predisposition to, or the incidence of, colorectal cancer in a sample comprising, consisting essentially of or consisting of detecting an epigenetic change in at least one gene selected from RASGRF2, SCNN1B, HOXD1, PLK2 and BHLHB9 wherein detection of the epigenetic change is indicative of a predisposition to, or the incidence of, colorectal cancer. The epigenetic change is methylation in certain embodiments.

The invention also provides a method for predicting the likelihood of successful treatment of colorectal cancer with a DNA demethylating agent and/or a DNA methyltransferase inhibitor and/or HDAC inhibitor comprising, consisting essentially of or consisting of detecting an epigenetic change in at least one gene selected from RASGRF2, SCNN1B, HOXD1, PLK2 and BHLHB9 in a sample, wherein detection of the epigenetic change is indicative that the likelihood of successful treatment is higher than if the epigenetic modification is not detected.

In a related aspect there is provided a method for predicting the likelihood of resistance to treatment of colorectal cancer with a DNA demethylating agent and/or DNA methyltransferase inhibitor and/or HDAC inhibitor comprising, consisting essentially of or consisting of detecting an epigenetic change in at least one gene selected from RASGRF2, SCNN1B, HOXD1, PLK2 and BHLHB9 in a sample, wherein detection of the epigenetic change is indicative that the likelihood of resistance to treatment is lower than if the epigenetic modification is not detected.

Also provided is a method of selecting a suitable treatment regimen for colorectal cancer comprising, consisting essentially of or consisting of detecting an epigenetic change in at least one gene selected from RASGRF2, SCNN1B, HOXD1, PLK2 and BHLHB9 in a sample, wherein detection of the epigenetic change results in selection of a DNA demethylating agent and/or a DNA methyltransferase inhibitor and/or a HDAC inhibitor for treatment and wherein if the epigenetic change is not detected, a DNA demethylating agent and/or a DNA methyltransferase inhibitor and/or a HDAC inhibitor is not selected for treatment.

The invention also provides a method of treating colorectal cancer in a subject comprising, consisting essentially of or consisting of administration of a DNA demethylating agent and/or a DNA demethylating agent and/or a DNA methyltransferase inhibitor wherein the subject has been selected for treatment on the basis of a method of the invention.

The invention also provides corresponding kits for carrying out the methods. In particular, a kit is provided for detecting a predisposition to, or the incidence of, colorectal cancer in a sample comprising, consisting essentially of or consisting of means for detecting an epigenetic change in at least one gene selected from RASGRF2, SCNN1B, HOXD1, PLK2 and BHLHB9.

In certain embodiments, the means for detecting methylation comprises, consists essentially of or consists of methylation specific PCR primers. Suitable primers are described herein and are represented as SEQ ID NOs 31-46 (as shown in Table 1 below)

Thus, in a further aspect the invention provides primer pairs for bisulphite genomic sequencing or methylation-specific PCR or RT-PCR selected from primers pairs comprising, consisting essentially of or consisting of the nucleotide sequences set forth in Table 1. The primer pairs are readily derivable from the information set forth in the table. More specifically, the invention provides bisulphite genomic sequencing primers or primers pairs comprising, consisting essentially of or consisting of the nucleotide sequences set forth as SEQ ID NOs 11-30 respectively. The invention also provides methylation-specific PCR primers or primer pairs selected from the primers or primer pairs comprising, consisting essentially of or consisting of the nucleotide sequences set forth as SEQ ID NOs 31-46. The invention also provides RT-PCR primers and primer pairs selected from the primers and primer pairs comprising, consisting essentially of or consisting of the nucleotide sequences set forth as SEQ ID NOs 47-54.

### BRIEF DESCRIPTION OF THE FIGURES

FIG. 1. Unmasking of epigenetically silenced genes using MeDIP.

A. Explanatory illustration of the MeDIP approach used. DNA methylation levels are calculated as the average of oligonucleotide ratios between immunoprecipitated 5-methylcytosine (IP α-5mC) vs Input. The confidence of binding calls is represented as a P-value. In the graph the probes marked with a red square (set p-value<0.001) have been considered to be potentially methylated (Bound).

B. Validation by real-time PCR of the enriched DNAs obtained from the MeDIP assays. Highly methylated promoters from the imprinted genes H19 and GPR109 and the tumor suppressor gene RARβ2 (hypermethylated in HCT-116) were selected as positive controls to measure the enrichment levels obtained after MeDIP. The graph shows a specific and efficient enrichment of methylated DNA over an unmethylated promoter (H3b) used as negative control.

C. Schematic strategy used to identify cancer-specific promoter hypermethylation in colon cancer cells using MeDIP.

D. Gene ontology categories of the 126 hypermethylated candidate genes obtained from the MeDIP approach. Ontology terms are shown on the Y axis; percentage of enrichment is graphed along the X axis.

FIG. 2. CpG island DNA methylation and expression analyses of the cancer specific hypermethylated genes found in the MeDIP approach.

A. Bisulfite genomic sequencing analyses of SCNN1B, RASGRF2, BHLHB9 and HOXD1 CpG island methylation status in HCT116, DKO and normal colon. CpG dinucleotides are represented as short vertical lines. The transcriptional start site is represented as a long black arrow and the location of bisulfite genomic sequencing PCR primers are indicated as white arrows. Ten single clones are represented for each sample. Presence of a methylated or unmethylated cytosine is indicated by a black or white square, respectively. The four CpG island are hypermethylated in HCT-116 cells, but unmethylated in DKO and normal colon.

B. Illustrative methylation-specific PCR analyses for SCNN1B, RASGRF2, BHLHB9 and HOXD1 gene in human normal colon samples (NC1-5). The presence of a PCR band under lanes M or U indicates methylated or unmethylated genes, respectively. In vitro methylated DNA (IVD) is used as positive control for methylated DNA. For BHLHB9 only colon samples from male donors were used. The four CpG island are unmethylated in normal colon.

C. Expression analyes for SCNN1B, RASGRF2, BHLHB9 and HOXD1 using reverse transcription PCR. Hypermethylated HCT-116 cells show loss of expression of the respective transcripts and restoration of expression is observed upon treatment with the demethylating agent 5-aza-2-deoxycytidine (DAC) and in DKO cells. The water reaction and normal colon are shown as negative and positive controls, respectively.

FIG. 3. CpG island DNA methylation and expression analyses of SCNN1B, RASGRF2, BHLHB9 and HOXD1 in human colon cancer cell lines, primary colorectal tumors and adenomas.

A. Methylation-specific PCR analysis of SCNN1B, RASGRF2, BHLHB9 and HOXD1 in human colon cancer cell lines. The presence of a PCR band under lanes M or U indicates methylated or unmethylated genes, respectively. Normal colon (NCOLON) and Normal lymphocytes (NL) are used as positive controls for unmethylated DNA and In vitro methylated DNA (IVD) is used as positive control for methylated DNA.

B. Expression analyses of SCNN1B, RASGRF2, BHLHB9 and HOXD1 by reverse transcription PCR in human colon cancer cell lines. The hypermethylated genes shown in FIG. 3A are not expressed, whilst unmethylated geres are expressed. Two illustrative normal colon cDNAs (N Colon1 and N Colon2) are shown as positive controls. GAPDH was used as internal control.

C. Representative methylation specific PCR analyses of SCNN1B, RASGRF2, BHLHB9 and HOXD1 in primary colorectal tumors (C1-5) showing unmethylated and methylated samples.

D. Representative methylation specific PCR analyses of SCNN1B, RASGRF2, BHLHB9 and HOXD1 in adenomas (AD1-5) showing unmethylated and methylated samples.

FIG. 4. Bisulfite genomic sequencing analyses of Elac2 (FIG. 4A), BID (FIG. 4B) and PLEKHE1 (FIG. 4C) CpG island methylation status in HCT116 and normal colon.

CpG dinucleotides are represented as short vertical lines. The transcriptional start site is represented as a long black arrow and the location of bisulfite genomic sequencing PCR primers are indicated as white arrows. Ten single clones are represented for each sample. Presence of a methylated or unmethylated cytosine is indicated by a black or white square, respectively. The three CpG islands are unmethylated in HCT-116 and normal colon.

FIG. 5. Bisulfite genomic sequencing analyses of DPPA4 (FIG. 5A) and BHLHB9 (FIG. 5B) CpG island methylation status in normal colon.

CpG dinucleotides are represented as short vertical lines. The transcriptional start site is represented as a long black arrow and the location of bisulfite genomic sequencing PCR primers are indicated as white arrows. Ten single clones are represented for each sample. Presence of a methylated or unmethylated cytosine is indicated by a black or white square, respectively. CpG island methylation is observed in all normal colon samples. BHLHB9 methylation is only observed in normal colon from female donors.

### DETAILED DESCRIPTION OF THE INVENTION

The present invention resulted from investigations into the extent of CpG island hypomethylation events in DKO cells and whether these cells could be used to find new genes with hypermethylation-associated inactivation in human cancer. In our early first preliminary genomic screening, we had observed CpG island hypomethylation events in putative tumor suppressor genes (7), but the current technology for large-scale epigenomic analyses (3) was not then available. The recent introduction of methylated DNA immunoprecipitation (MeDIP) technology combined with comprehensive gene promoter arrays (8-10) prompted us to reinvestigate the DKO cells using this new epigenomic tool. Our results demonstrate that cancer cells lacking DNMT1 and DNMT3b undergo significant CpG island hypomethylation events that identify new putative tumor suppressor genes undergoing methylation-associated silencing in human cancer. These data contribute to a more complete map of the DNA hypermethylome of malignant cells and provide new hypermethylated markers for putative translational use in colorectal cancer patients.

Thus, in a first aspect the invention provides a method of identifying nucleic acid molecules differentially methylated in a disease comprising, consisting essentially of or consisting of

(a) incubating fragmented DNA, from a disease cell, with a reagent which specifically binds to methylated DNA to thus concentrate methylated DNA fragments
(b) incubating fragmented DNA, from a disease cell related to the disease cell utilised in step (a) in which DNA methyltransferase expression and/or activity has been inhibited, with a reagent which specifically binds to methylated DNA to thus concentrate methylated DNA fragments
(c) comparing the methylated DNA fragments obtained in steps (a) and (b) to identify nucleic acid molecules differentially methylated in the disease.

The nucleic acid molecules identified according to the methods generally comprise genes. In particular, the 5′ region of genes, typically including the promoter region, are often differentially methylated (generally hypermethylated) in disease cells. However, the differentially methylated nucleic acid molecules may be derived from any genomic DNA found within the tested cells.

In certain embodiments, the differential methylation comprises, consists essentially of or consists of increased methylation in the disease cell as compared to the disease cell in which DNA methyltransferase expression and/or activity has been inhibited.

The methods of the invention may be used to investigate any disease condition in which it may be suspected that cells are differentially methylated in the diseased state. Thus, the “disease cell” is a cell representative of the disease (for example a cell taken from disease tissue or an appropriate cell line derived from a disease). In some embodiments, the disease comprises, consists essentially of or consists of a cell proliferative disorder. In these embodiments, the disease cell comprises, consists essentially of or consists of a cell representative of the cell proliferative disorder. The cells may be taken from disease tissue or they may be suitable cell lines representative of the disorder. In specific embodiments, the cell proliferative disorder comprises, consists essentially of or consists of cancer. Thus, in these embodiments, the disease cell comprises, consists essentially of or consists of a cell representative of the cancer. The cancer comprises, consists essentially of or consists of colorectal cancer in specific embodiments (and thus the disease cell comprises, consists essentially of or consists of a cell representative of the colorectal cancer).

A particularly useful disease cell in the context of the present invention comprises, consists essentially of or consists of a HCT-116 cell. HCT-116 is a well-known cell line representative of colorectal cancer. In related embodiments, the disease cell related to the disease cell utilised in step (a) in which DNA methyltransferase expression and/or activity has been inhibited comprises, consists essentially of or consists of a DKO cell. The DKO cell is also known in the art and represents a derivative of the HCT-116 cell in which both DNMT1 and DNMT3b genes have been knocked out.

As indicated, the methods are based upon fragmented (genomic) DNA. In order to improve the effectiveness of the downstream steps of the method the fragments of DNA may be between around 100 and 1000 base pairs (bp) or nucleotides, such as between around 200 or 300 bp and 600 or 800 bp. In certain embodiments, the method further comprises, consists essentially of or consists of fragmentation of DNA from the disease cell. This may be done as a preliminary step in the methods to produce the fragmented DNA which is the incubated with an appropriate reagent (as discussed herein). Similarly, the methods may also further comprise, consist essentially of or consist of fragmentation of DNA from the disease cell in which DNA methyltransferase expression and/or activity has been inhibited. Any suitable method of fragmentation, as would be known to those skilled in the art, may be employed. The methods may be physical or chemical methods. Specific examples include sonication or restriction digestion.

The methods involve specific capture of methylated DNA fragments. This specific binding achieves concentration of the methylated DNA fragments by separating methylated fragments from those which are unmethylated. Any suitable reagent may be employed for this purpose. The reagent may be employed under any suitable experimental conditions. Thus, by “incubating” is meant bringing the fragmented DNA (in both steps (a) and (b)) into contact with the reagent under conditions which permit methylated DNA fragments in the sample to be bound by the reagent. Suitable conditions are described in the experimental section herein, but it would be apparent to the skilled person that such conditions are not limiting. In particular, a range of temperatures and times of incubation may be employed, and preferred conditions found through routine experimentation. Suitable buffers may be employed, in which the reagent and DNA are stable. Such buffers, including standard immunoprecipitation buffers, are well known in the art and commercially available. In certain embodiments the reagent which specifically binds to methylated DNA comprises, consists essentially of or consists of an antibody, or a derivative thereof that retains specific binding activity.

By specific binding activity is meant the ability to specifically bind to methylated DNA. Thus, such a reagent does not bind, or does not bind to a significant degree, to unmethylated DNA. Any antibody or derivative may be employed. Thus, the antibody may be a monoclonal or polyclonal antibody. The derivative of the antibody that retains specific binding activity comprises, consists essentially of or consists of a humanized version of a non-human antibody, a heavy chain antibody, a single domain antibody, a nanobody, a Fab fragment or scFv in certain embodiments. Numerous techniques are available for producing antibodies and their derivatized forms, as would be well known to one skilled in the art. The antibody or antibody derivative comprises, consists essentially of or consists of an antibody directed against 5-methyl-cytosine or a derivative thereof that retains specific binding activity in certain embodiments. Antibodies that specifically bind to 5-methyl-cytosine and do not bind, or do not significantly bind, to unmethylated cytosine are known in the art and commercially available (e.g. from Eurogentec). Thus, the methods of the invention may involve concentration of the methylated DNA fragments through immunocapture of methylated DNA fragments.

The methods of the invention may be utilised to identify nucleic acid molecules which are aberrantly methylated in a disease. As is known in the art, aberrant methylation, generally increased or hypermethylation, is linked to the incidence of certain conditions. Conditions include cellular proliferative disorders such as cancer and in particular colorectal cancer, as discussed above. The methods may thus be used to identify novel markers whose methylation status is linked to the incidence of the disease. In particular, markers which become hypermethylated in the diseased state may be discovered, as discussed and shown experimentally herein.

As indicated, the DNA utilised in step (b) of the method is taken from a derivative of the disease cell in which DNA methyltransferase (DNMT) expression and/or activity has been inhibited. Any DNA methyltransferase function may be inhibited in the cell provided it gives the necessary corresponding decrease in methylation of genomic DNA, thus allowing DNA methylated in the disease cell to be identified. In specific embodiments, DNMT1 and/or DNMT3b expression and/or activity has been inhibited. These two DNMTs have been shown to be particularly useful in the context of preparing cells derivatized from the disease cells for use in the methods of the invention. DNA methyltransferase expression and/or activity may be inhibited by any suitable mechanism. For example, DNA methyltransferase expression and/or activity may be inhibited through gene knockout of one or more DNA methyltransferase genes. Suitable techniques for inhibiting a given enzyme expression activity or expression are known in the art and include recombination based techniques, antisense, RNA interference (e.g. mediated through siRNA), mutagenesis, use of specific inhibitors etc. In specific embodiments, gene knockout is achieved through homologous recombination.

In specific embodiments of the methods, step (c) comprises, consists essentially of or consists of differentially labelling the methylated DNA fragments obtained in steps (a) and (b) and hybridizing the methylated DNA fragments to a microarray to identify nucleic acid molecules differentially methylated in the disease The present invention is based, in part, upon the combination of MeDIP and microarray technology to investigate methylation of DNA linked to disease. In certain embodiments, the microarray comprises, consists essentially of or consists of a plurality of promoter nucleotide sequences. As discussed herein, and as is known in the art, functionally relevant cytosine methylation typically occurs in CpG islands, often found in or around the promoter region of genes. Any microarray fit for purpose may be employed. Suitable microarrays are commercially available and comprise, consist essentially of or consist of the Human Proximal Promoter Array 44K (Agilent Technologies, Palo Alto, Calif.).

The differential labelling of the two source of methylated DNA may be achieved by any suitable means. In certain embodiments, the labels comprise fluorescent labels. Suitable fluorescent labels are well known in the art and commercially available. For example, the fluorescent labels may comprise Cy5 and Cy3 fluorophores respectively.

In other embodiments, the concentration of methylated DNA fragments obtained in each of steps (a) and (b) is quantified relative to the overall input of fragmented DNA in each case. This allows comparisons between the two samples to be made more readily. The methylated DNA fragments and overall input of fragmented DNA for each of steps (a) and (b) may be differentially labelled and hybridized to a microarray (in each case) to allow identification of methylated DNA fragments concentrated through incubation with the reagent which specifically binds to methylated DNA. Again, the labels may comprise fluorescent labels, in particular the fluorescent labels may comprise Cy5 and Cy3 fluorophores respectively. Any microarray fit for purpose may be employed. Suitable microarrays are commercially available and comprise, consist essentially of or consist of the Human Proximal Promoter Array 44K (Agilent Technologies, Palo Alto, Calif.).

In these microarray based methods, step (c) comprises, consists essentially of or consists of comparing the hybridization patterns of the respective microarrays obtained for the methylated DNA fragments and overall input of fragmented DNA for each of steps (a) and (b) to identify nucleic acid molecules differentially methylated in the disease in some embodiments. By appropriate labelling of the respective DNA components, suitable software may be employed to assess the results of the hybridization experiments.

The methods of the invention may involve further characterisation of nucleic acid molecules identified as being differentially methylated in the disease by determining the presence or absence of a CpG island in the nucleotide sequence. As discussed herein, since aberrant methylation linked to disease status is often found in specific genes—for example methylation of tumour suppressor genes may be linked to the incidence of cancer—the presence or absence of a CpG island at or near the 5′ end of the nucleotide sequence may be determined. Thus, the promoter regions of genes of interest may be assessed to determine if a CpG island, susceptible to hypermethylation, is in fact present. Any suitable technique may be employed. Sequencing may be utilised, although a number of accurate in silico techniques are now available, which are perhaps more convenient.

The methods of the invention may, in certain embodiments, (further) comprise, consist essentially of or consist of determining the methylation status of the CpG island of the nucleic acid molecules which include a CpG island from a disease cell to identify nucleic acid molecules which are methylated in the disease cell. The methods of the invention may, in certain embodiments, (further) comprise, consist essentially of or consist of determining the methylation status of the CpG island of the nucleic acid molecules which include a CpG island from both a disease cell and a disease cell in which DNA methyltransferase expression and/or activity has been inhibited to identify nucleic acid molecules which are methylated in the disease cell but unmethylated or methylated to a lesser extent in the disease cell in which DNA methyltransferase expression and/or activity has been inhibited.

The methods may, in certain embodiments, further comprise, consist essentially of or consist of determining the methylation status of the CpG island of the nucleic acid molecules which include a CpG island in a non-disease cell. A lack of methylation or a lesser degree of methylation in the non-disease cell (as compared to the level of methylation in the disease cell) indicates that the nucleic acid molecule is methylated as an indicator of the disease. In certain embodiments, the non-disease cell is derived from non-disease tissue of the same type as the tissue from which the disease cell is derived. This allows a direct comparison to be made to determine whether the methylation is linked to the incidence of disease. In specific embodiments, the non-disease cell is derived from colon and/or rectal and/or appendix tissue. This is useful when identifying markers linked to diseases of these tissues, such as colorectal cancer as exemplified herein.

Determination of the methylation status may be achieved through any suitable means. Suitable examples include bisulphite genomic sequencing and/or by methylation specific PCR. Various techniques for assessing methylation status are known in the art and can be used in conjunction with the present invention: sequencing, methylation-specific PCR (MS-PCR), melting curve methylation-specific PCR (McMS-PCR), MLPA with or without bisulphite treatment, QAMA (Zeschnigk et al, 2004), MSRE-PCR (Melnikov et al, 2005), MethyLight (Eads et al., 2000), ConLight-MSP (Rand et al., 2002), bisulphite conversion-specific methylation-specific PCR (BS-MSP) (Sasaki et al., 2003), COBRA (which relies upon use of restriction enzymes to reveal methylation dependent sequence differences in PCR products of sodium bisulphite—treated DNA), methylation-sensitive single-nucleotide primer extension conformation (MS-SNuPE), methylation-sensitive single-strand conformation analysis (MS-SSCA), Melting curve combined bisulphite restriction analysis (McCOBRA) (Akey et al., 2002), PyroMethA, HeavyMethyl (Cottrell et al. 2004), MALDI-TOF, MassARRAY, Quantitative analysis of methylated alleles (QAMA), enzymatic regional methylation assay (ERMA), QBSUPT, MethylQuant, Quantitative PCR sequencing and oligonucleotide-based microarray systems, Pyrosequencing, Meth-DOP-PCR. A review of some useful techniques for DNA methylation analysis is provided in Nucleic acids research, 1998, Vol. 26, No. 10, 2255-2264, Nature Reviews, 2003, Vol. 3, 253-266; Oral Oncology, 2006, Vol. 42, 5-13, which references are incorporated herein in their entirety.

Techniques for assessing methylation status are based on distinct approaches. Some include use of endonucleases. Such endonucleases may either preferentially cleave methylated recognition sites relative to non-methylated recognition sites or preferentially cleave non-methylated relative to methylated recognition sites. Some examples of the former are Acc III, Ban I, BstN I, Msp I, and Xma I. Examples of the latter are Acc II, Ava I, BssH II, BstU I, Hpa II, and Not I. Differences in cleavage pattern are indicative for the presence or absence of a methylated CpG dinucleotide. Cleavage patterns can be detected directly, or after a further reaction which creates products which are easily distinguishable. Means which detect altered size and/or charge can be used to detect modified products, including but not limited to electrophoresis, chromatography, and mass spectrometry.

Alternatively, the identification of methylated CpG dinucleotides may utilize the ability of the methyl binding domain (MBD) of the MeCP2 protein to selectively bind to methylated DNA sequences (Cross et al, 1994; Shiraishi et al, 1999). The MBD may also be obtained from MBP, MBP2, MBP4, poly-MBD (Jorgensen et al., 2006) or from reagents such as antibodies binding to methylated nucleic acid. The MBD may be immobilized to a solid matrix and used for preparative column chromatography to isolate highly methylated DNA sequences. Variant forms such as expressed His-tagged methyl-CpG binding domain may be used to selectively bind to methylated DNA sequences. Eventually, restriction endonuclease digested genomic DNA is contacted with expressed His-tagged methyl-CpG binding domain. Other methods are well known in the art and include amongst others methylated-CpG island recovery assay (MIRA). Another method, MB-PCR, uses a recombinant, bivalent methyl-CpG-binding polypeptide immobilized on the walls of a PCR vessel to capture methylated DNA and the subsequent detection of bound methylated DNA by PCR.

Further approaches for detecting methylated CpG dinucleotide motifs use chemical reagents that selectively modify either the methylated or non-methylated form of CpG dinucleotide motifs. Suitable chemical reagents include hydrazine and bisulphite ions. The methods of the invention preferably use bisulphite ions. The bisulphite conversion relies on treatment of DNA samples with sodium bisulphite which converts unmethylated cytosine to uracil, while methylated cytosines are maintained (Furuichi et al., 1970). This conversion finally results in a change in the sequence of the original DNA. It is general knowledge that the resulting uracil has the base pairing behaviour of thymidine which differs from cytosine base pairing behaviour. This makes the discrimination between methylated and non-methylated cytosines possible. Useful conventional techniques of molecular biology and nucleic acid chemistry for assessing sequence differences are well known in the art and explained in the literature. See, for example, Sambrook, J., et al., Molecular cloning: A laboratory Manual, (2001) 3rd edition, Cold Spring Harbor, N.Y.; Gait, M. J. (ed.), Oligonucleotide Synthesis, A Practical Approach, IRL Press (1984); Hames B. D., and Higgins, S. J. (eds.), Nucleic Acid Hybridization, A Practical Approach, IRL Press (1985); and the series, Methods in Enzymology, Academic Press, Inc.

Some techniques use primers for assessing the methylation status at CpG dinucleotides. Two approaches to primer design are possible. Firstly, primers may be designed that themselves do not cover any potential sites of DNA methylation. Sequence variations at sites of differential methylation are located between the two primers and visualisation of the sequence variation requires further assay steps. Such primers are used in bisulphite genomic sequencing, COBRA, Ms-SnuPE and several other techniques. Secondly, primers may be designed that hybridize specifically with either the methylated or unmethylated version of the initial treated sequence. After hybridization, an amplification reaction can be performed and amplification products assayed using any detection system known in the art. The presence of an amplification product indicates that a sample hybridized to the primer. The specificity of the primer indicates whether the DNA had been modified or not, which in turn indicates whether the DNA had been methylated or not. If there is a sufficient region of complementarity, e.g., 12, 15, 18, or 20 nucleotides, to the target, then the primer may also contain additional nucleotide residues that do not interfere with hybridization but may be useful for other manipulations. Examples of such other residues may be sites for restriction endonuclease cleavage, for ligand binding or for factor binding or linkers or repeats. The oligonucleotide primers may or may not be such that they are specific for modified methylated residues.

A further way to distinguish between modified and unmodified nucleic acid is to use oligonucleotide probes. Such probes may hybridize directly to modified nucleic acid or to further products of modified nucleic acid, such as products obtained by amplification. Probe-based assays exploit the oligonucleotide hybridisation to specific sequences and subsequent detection of the hybrid. There may also be further purification steps before the amplification product is detected e.g. a precipitation step. Oligonucleotide probes may be labelled using any detection system known in the art. These include but are not limited to fluorescent moieties, radioisotope labelled moieties, bioluminescent moieties, luminescent moieties, chemiluminescent moieties, enzymes, substrates, receptors, or ligands.

In the MSP approach, DNA may be amplified using primer pairs designed to distinguish methylated from unmethylated DNA by taking advantage of sequence differences as a result of sodium-bisulphite treatment (Herman et al., 1996; and WO 97/46705). For example, bisulphite ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulphite-modified DNA, whereas an oligonucleotide primer containing the guanine bases would hybridize to the non-modified (methylated) cytosine residues in the DNA. Amplification using a DNA polymerase and a second primer yield amplification products which can be readily observed, which in turn indicates whether the DNA had been methylated or not. Whereas PCR is a preferred amplification method, variants on this basic technique such as nested PCR and multiplex PCR are also included within the scope of the invention.

As mentioned earlier, a preferred embodiment for assessing the methylation status of the relevant gene requires amplification to yield amplification products. The presence of amplification products may be assessed directly using methods well known in the art. They simply may be visualized on a suitable gel, such as an agarose or polyacrylamide gel. Detection may involve the binding of specific dyes, such as ethidium bromide, which intercalate into double-stranded DNA and visualisation of the DNA bands under a UV illuminator for example. Another means for detecting amplification products comprises hybridization with oligonucleotide probes. Alternatively, fluorescence or energy transfer can be measured to determine the presence of the methylated DNA.

A specific example of the MSP technique is designated real-time quantitative MSP (QMSP), and permits reliable quantification of methylated DNA in real time or at end point. Real-time methods are generally based on the continuous optical monitoring of an amplification procedure and utilise fluorescently labelled reagents whose incorporation in a product can be quantified and whose quantification is indicative of copy number of that sequence in the template. One such reagent is a fluorescent dye, called SYBR Green I that preferentially binds double-stranded DNA and whose fluorescence is greatly enhanced by binding of double-stranded DNA. Alternatively, labeled primers and/or labeled probes can be used for quantification. They represent a specific application of the well known and commercially available real-time amplification techniques such as TAQMAN®, MOLECULAR BEACONS®, AMPLIFLUOR® and SCORPION® DzyNA®, Plexor™ etc. In the real-time PCR systems, it is possible to monitor the PCR reaction during the exponential phase where the first significant increase in the amount of PCR product correlates to the initial amount of target template.

Real-Time PCR detects the accumulation of amplicon during the reaction. Real-time methods do not need to be utilised, however. Many applications do not require quantification and Real-Time PCR is used only as a tool to obtain convenient results presentation and storage, and at the same time to avoid post-PCR handling. Thus, analyses can be performed only to confirm whether the target DNA is present in the sample or not. Such end-point verification is carried out after the amplification reaction has finished. This knowledge can be used in a medical diagnostic laboratory to detect a predisposition to, or the incidence of, cancer in a patient. End-point PCR fluorescence detection techniques can use the same approaches as widely used for Real Time PCR. For example, <<Gene>> detector allows the measurement of fluorescence directly in PCR tubes.

In real-time embodiments, quantitation may be on an absolute basis, or may be relative to a constitutively methylated DNA standard, or may be relative to an unmethylated DNA standard. Methylation status may be determined by using the ratio between the signal of the marker under investigation and the signal of a reference gene where methylation status is known (such as β-actin for example), or by using the ratio between the methylated marker and the sum of the methylated and the non-methylated marker. Alternatively, absolute copy number of the methylated marker gene can be determined.

Suitable controls may need to be incorporated in order to ensure the method chosen is working correctly and reliably. Suitable controls may include assessing the methylation status of a gene known to be methylated. This experiment acts as a positive control to ensure that false negative results are not obtained. The gene may be one which is known to be methylated in the sample under investigation or it may have been artificially methylated, for example by using a suitable methyltransferase enzyme, such as SssI methyltransferase.

Additionally or alternatively, suitable negative controls may be employed with the methods of the invention. Here, suitable controls may include assessing the methylation status of a gene known to be unmethylated or a gene that has been artificially demethylated. This experiment acts as a negative control to ensure that false positive results are not obtained.

Whilst PCR is the preferred nucleic acid amplification technique, other amplification techniques may also be utilised to detect the methylation status of the concerned gene. Such amplification techniques are well known in the art, and include methods such as NASBA (Compton, 1991), 3SR (Fahy et al., 1991) and Transcription Mediated Amplification (TMA). Other suitable amplification methods include the ligase chain reaction (LCR) (Barringer et al, 1990), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO 90/06995), invader technology, strand displacement technology, and nick displacement amplification (WO 2004/067726). This list is not intended to be exhaustive; any nucleic acid amplification technique may be used provided the appropriate nucleic acid product is specifically amplified. Thus, these amplification techniques may be tied in to MSP and/or bisulphite sequencing techniques for example.

In certain embodiments, the methods of the invention may further comprise, consist essentially of or consist of determining the effect of methylation on expression of the nucleic acid molecule by comparing gene expression in the disease cell and disease cell in which DNA methyltransferase expression and/or activity has been inhibited. This is one manner in which to confirm that the differential methylation is functionally relevant. In specific embodiments, expression is determined at the RNA level. Any suitable technique may be employed. In certain embodiments, expression at the RNA level is determined by reverse transcriptase polymerase chain reaction (RT-PCR). In alternative embodiments, expression is determined at the protein level. Again, any suitable technique may be employed such as western blotting, ELISA etc.

As a further confirmation of the functional relevance of the methylation, the methods of the invention may further comprise, consist essentially of or consist of determining whether use of a demethylating agent can restore expression of the nucleic acid molecule in the disease cell. If the result is positive, this indicates that the methylation is the cause of the loss of expression. Any suitable demethylating agent may be employed, of which many are known. In specific embodiments, the demethylating agent comprises, consists essentially of or consists of 5-aza-2-deoxycytidine.

The methods of the invention may be utilised to identify candidate tumour suppressor genes. Thus, the methods of the invention are particularly valuable for identifying new markers whose methylation status is linked to disease. Methylation of tumour suppressor genes has been shown to be linked to the incidence of cancer in certain cases and the methods of the invention may assist in discovering further tumour suppressor genes. In agreement with this, the methods of the invention have indeed resulted in identification of a number of novel markers shown to be methylated in colorectal cancer.

Thus, according to a further aspect, the invention provides a method of detecting a predisposition to, or the incidence of, colorectal cancer in a sample comprising, consisting essentially of or consisting of detecting an epigenetic change in at least one gene selected from RASGRF2, SCNN1B, HOXD1, PLK2 and BHLHB9 wherein detection of the epigenetic change is indicative of a predisposition to, or the incidence of, colorectal cancer.

“RASGRF2, SCNN1B, HOXD1, PLK2 and BHLHB9” is the standard nomenclature for Ras protein-specific guanine nucleotide-releasing factor 2 (RASGRF2, Accession number: AF023130 and NM_{—}006909), sodium channel, nonvoltage-gated 1, beta (SCNN1B, Accession number: X87159), homeobox D1 (HOXD1, Accession number: NM_{—}024501), polo-like kinase 2 (PLK2, Accession number: NM_{—}006622) and basic helix-loop-helix domain containing, class B, 9 (BHLHB9, Accession number: AB051488 and NM_{—}030639).

By “gene” is meant the specific known gene in question. It may also relate to any gene which is taken from the family to which the named “gene” belongs and includes according to all aspects of the invention not only the particular sequences found in the publicly available database entries, but also encompasses transcript and nucleotide variants of these sequences, with the proviso that methylation or another epigenetic modification of the gene is linked to the incidence of colorectal cancer.

These methods of the invention may be ex vivo or in vitro methods carried out on a test sample. The methods may be non-invasive. The methods may be used to identify any stage of colorectal cancer, including pre-malignancies such as adenomas right through to carcinomas.

The “sample” in which the epigenetic change of the at least one gene selected from RASGRF2, SCNN1B, HOXD1, PLK2 and BHLHB9 is detected may comprise, consist essentially of or consist of a tissue sample, a faecal sample or a blood sample. In specific embodiments, the tissue sample comprises, consists essentially of or consists of a colon and/or rectum and/or appendix sample. However, the sample may be from any representative tissue sample, body fluid, body fluid precipitate or lavage specimen, as required. The sample may be obtained from a human subject. Test samples for diagnostic, prognostic, or personalised medicinal uses can be obtained from surgical samples, such as biopsies or fine needle aspirates, from paraffin embedded tissues, from frozen tumor tissue samples, from fresh tumor tissue samples, from a fresh or frozen body fluid, for example. Non-limiting examples include whole blood, bone marrow, cerebral spinal fluid, peritoneal fluid, pleural fluid, lymph fluid, serum, plasma, urine, chyle, stool, ejaculate, sputum, nipple aspirate, saliva, swabs specimen, wash or lavage fluid and/or brush specimens.

These methods may also include the step of obtaining the test sample, in certain embodiments. The tissue sample or liquid sample comprising the nucleic acid may be lysed or need to be concentrated to create a mixture of biological compounds comprising nucleic acids and other components. Alternatively, the nucleic acid may need to be cleared of proteins or other contaminants, e.g. by treatment with proteinase K. Procedures for lysing or concentrating biological samples are known by the person skilled in the art and can be chemical, enzymatic or physical in nature. A combination of these procedures may be applicable as well. For instance, lysis may be performed using ultrasound, high pressure, shear forces, alkali, detergents or chaotropic saline solutions, or proteases or lipases. For the lysis procedure to obtain nucleic acids, or concentrating nucleic acid from samples, reference may be made to Sambrook, J., et al., Molecular cloning: A Laboratory Manual, (2001) 3rd edition, Cold Spring Harbor Laboratory Press, Cold Spring Harbor, N.Y. Ausubel, F. M., et al., Current Protocols in Molecular Biology (1987), J. Wiley and Sons, New York.

The test sample is generally obtained from a (human) subject suspected of being tumorigenic. Alternatively the test sample is obtained from a subject undergoing routine examination and not necessarily being suspected of having a disease. Thus patients at risk can be identified before the disease has a chance to manifest itself in terms of symptoms identifiable in the patient. Alternatively the sample is obtained from a subject undergoing treatment, or from patients being checked for recurrence of disease.

In specific embodiments, the at least one gene is BHLHB9 and the sample is from a male subject. BHLHB9 is located on the X chromosome and is thus generally not methylated in normal males, in which X chromosome inactivation does not occur. Thus, detection of hypermethylation of this gene in males was found to be specifically associated with the incidence of colorectal cancer, as described herein.

In further embodiments, the method comprises, consists essentially of or consists of detecting an epigenetic change in a panel of genes comprising, consisting essentially of or consisting of at least two, three, four or five of the genes (RASGRF2, SCNN1B, HOXD1, PLK2 and BHLHB9), wherein detection of an epigenetic change in at least one of the genes in the panel is indicative of a predisposition to, or the incidence of, colorectal cancer. The panel of genes thus may comprise, consist essentially of or consist of two, three, four or five genes. In specific embodiments, the panel of genes comprises, consists essentially of or consists of RASGRF2 and SCNN1B. The detection of an epigenetic change in each of the panel of genes may be carried out in a single reaction. This is possible for example through multiplexing experiments which are known in the art.

In certain embodiments of these methods, the epigenetic change is methylation. Thus, aberrant methylation, or “hypermethylation”, of the gene(s) may be detected. This is typically measured in one or more CpG islands, often located in or around the promoter regions of the relevant genes. Methylation may be determined using any suitable technique, as discussed extensively above. In specific embodiments, methylation specific PCR/amplification is utilised. This may be carried out in real time or at end point. The real time or end point PCR/amplification may involve use of hairpin primers (Amplifluor), hairpin probes (Molecular Beacons), hydrolytic probes (Taqman), FRET probe pairs (Lightcycler), primers incorporating a hairpin probe (Scorpion), fluorescent dyes (SYBR Green etc.), primers incorporating the complementary sequence of a DNAzyme and a cleavable fluorescent DNAzyme substrate or oligonucleotide blockers, for example.

In specific embodiments, the method utilises methylation-specific PCR primers or primer pairs selected from the primers or primer pairs comprising, consisting essentially of or consisting of the nucleotide sequences set forth as SEQ ID NOs 31-46 (see Table 1).

Alternatively, methylation is determined using bisulphite sequencing. At least one CpG island in the at least one gene may be sequenced using this method. The CpG island may be found in the promoter and/or 5′ untranslated region and/or first exon of the at least one gene. This method may utilise bisulphite genomic sequencing primers or primers pairs comprising, consisting essentially of or consisting of the nucleotide sequences set forth as SEQ ID NOs 11-30 respectively (see Table 1).

In a related aspect, the invention provides a method for predicting the likelihood of successful treatment of colorectal cancer with a DNA demethylating agent and/or a DNA methyltransferase inhibitor and/or HDAC inhibitor comprising, consisting essentially of or consisting of detecting an epigenetic change in at least one gene selected from RASGRF2, SCNN1B, HOXD1, PLK2 and BHLHB9 in a sample, wherein detection of the epigenetic change is indicative that the likelihood of successful treatment is higher than if the epigenetic modification is not detected.

Similarly, the invention provides a method for predicting the likelihood of resistance to treatment of colorectal cancer with a DNA demethylating agent and/or DNA methyltransferase inhibitor and/or HDAC inhibitor comprising, consisting essentially of or consisting of detecting an epigenetic change in at least one gene selected from RASGRF2, SCNN1B, HOXD1, PLK2 and BHLHB9 in a sample, wherein detection of the epigenetic change is indicative that the likelihood of resistance to treatment is lower than if the epigenetic modification is not detected.

Also, the invention provides a method of selecting a suitable treatment regimen for colorectal cancer comprising, consisting essentially of or consisting of detecting an epigenetic change in at least one gene selected from RASGRF2, SCNN1B, HOXD1, PLK2 and BHLHB9 in a sample, wherein detection of the epigenetic change results in selection of a DNA demethylating agent and/or a DNA methyltransferase inhibitor and/or a HDAC inhibitor for treatment and wherein if the epigenetic change is not detected, a DNA demethylating agent and/or a DNA methyltransferase inhibitor and/or a HDAC inhibitor is not selected for treatment.

For each of these additional aspects, the embodiments and optional features of the methods of the invention apply mutatis mutandis and are not repeated for reasons of conciseness. Thus, all methods of detecting an epigenetic change in the at least one gene may be employed appropriately.

In all of these methods, the epigenetic change may measured indirectly at the level of gene expression. This may be at the level of mRNA. Expression at the level of mRNA may be measured using any suitable method. Examples include reverse transcriptase polymerase chain reaction (RT-PCR) or an equivalent amplification technique. Such methods may utilise RT-PCR primers and primer pairs selected from the primers and primer pairs comprising, consisting essentially of or consisting of the nucleotide sequences set forth as SEQ ID NOs 47-54 (see Table 1).

The RT-PCR or an equivalent amplification technique may be carried out in real time or at end point (as discussed herein). The real time or end point PCR or an equivalent amplification technique may involve use of hairpin primers (Amplifluor), hairpin probes (Molecular Beacons), hydrolytic probes (Taqman), FRET probe pairs (Lightcycler), primers incorporating a hairpin probe (Scorpion), fluorescent dyes (SYBR Green etc.), primers incorporating the complementary sequence of a DNAzyme and a cleavable fluorescent DNAzyme substrate or oligonucleotide blockers in certain embodiments. Alternatively, gene expression may be determined at the protein level. Again, any suitable technique may be employed.

In a further related aspect, the invention provides a method of treating colorectal cancer in a subject comprising, consisting essentially of or consisting of administration of a DNA demethylating agent and/or a DNA demethylating agent and/or a DNA methyltransferase inhibitor wherein the subject has been selected for treatment on the basis of a method of the invention. Thus identifying an epigenetic change in at least one gene selected from RASGRF2, SCNN1B, HOXD1, PLK2 and BHLHB9 in a sample may be used in order to direct treatment of the subject (from which the sample was taken).

The invention also relates to corresponding kits for carrying out the methods of the invention. Thus, the invention provides a kit for detecting a predisposition to, or the incidence of, colorectal cancer in a sample comprising, consisting essentially of or consisting of means for detecting an epigenetic change in at least one gene selected from RASGRF2, SCNN1B, HOXD1, PLK2 and BHLHB9. In certain embodiments, the at least one gene is selected from RASGRF2 and SCNN1B. The kit may comprise, consist essentially of or consist of means for detecting an epigenetic change in a panel of genes comprising, consisting essentially of or consisting of at least two, three, four or five of the genes (RASGRF2, SCNN1B, HOXD1, PLK2 and BHLHB9), wherein detection of an epigenetic change in at least one of the genes in the panel is indicative of a predisposition to, or the incidence of, colorectal cancer. The panel of genes comprises, consists essentially of or consists of two, three, four or five genes in certain embodiments.

In some embodiments, the means for detecting an epigenetic change in the panel of genes enable the detection to be carried out in a single reaction. Thus the means may permit multiplexing.

As discussed in respect of the methods of the invention, the epigenetic change is preferably methylation. The kit may permit aberrant methylation (hypermethylation) within at least one CpG island to be detected. The CpG island may be found in the promoter and/or 5′ untranslated region and/or first exon of the gene(s).

In certain embodiments, the means for detecting methylation comprises, consists essentially of or consists of methylation specific PCR primers. The means may comprise any primer type which permits the methylation status of the at least one gene to be directly determined. In certain embodiments, the kit further comprises, consists essentially of or consists of means for carrying out the methylation specific PCR or an equivalent amplification technique in real time or at end point. The means for carrying out the methylation specific PCR or an equivalent amplification technique in real time or at end point comprises, consists essentially of or consists of hairpin primers (Amplifluor), hairpin probes (Molecular Beacons), hydrolytic probes (Taqman), FRET probe pairs (Lightcycler), primers incorporating a hairpin probe (Scorpion), fluorescent dyes (SYBR Green etc.), primers incorporating the complementary sequence of a DNAzyme and a cleavable fluorescent DNAzyme substrate or oligonucleotide blockers in specific embodiments.

In specific embodiments, the methylation-specific PCR primers or primer pairs are selected from the primers or primer pairs comprising, consisting essentially of or consisting of the nucleotide sequences set forth as SEQ ID NOs 31-46 (see table 1).

In alternative embodiments, methylation is determined using bisulphite sequencing and thus the means for detecting an epigenetic change in at least one gene selected from RASGRF2, SCNN1B, HOXD1, PLK2 and BHLHB9 comprises, consists essentially of or consists of primers for bisulphite sequencing. In specific embodiments, the primers for bisulphite sequencing are bisulphite genomic sequencing primers or primers pairs comprising, consisting essentially of or consisting of the nucleotide sequences set forth as SEQ ID NOs 11-30 respectively (see table 1).

In certain embodiments, the kit further comprises, consists essentially of or consists of a reagent which selectively modifies unmethylated cytosine residues in the DNA contained in the sample to produce detectable modified residues but which does not modify methylated cytosine residues. Such a reagent is required for detection techniques such as MSP, MethyLight and bisulphite sequencing. In specific embodiments, the reagent comprises, consists essentially of or consists of a bisulphite reagent. Bisulphite reagents convert unmethylated cytosine residues to uracil, whereas methylated cytosine residues remain unconverted. Any suitable bisulphite reagent may be employed. In specific embodiments, the bisulphite reagent comprises, consists essentially of or consists of sodium bisulphite.

In some embodiments, wherein the epigenetic change is measured indirectly at the level of gene expression, the means for detecting the epigenetic change may be means for determining gene expression of the at least one gene. In certain embodiments, gene expression is measured at the level of mRNA and thus the kit incorporates appropriate primers and/or probes. Where mRNA is measured using reverse transcriptase polymerase chain reaction (RT-PCR) or an equivalent amplification technique suitable RT-PCR primers may be included in the kits of the invention. In specific embodiments, the kit comprises RT-PCR primers and primer pairs selected from the primers and primer pairs comprising, consisting essentially of or consisting of the nucleotide sequences set forth as SEQ ID NOs 47-54 (see table 1). In further embodiments, the kit further comprises, consists essentially of or consists of hairpin primers (Amplifluor), hairpin probes (Molecular Beacons), hydrolytic probes (Taqman), FRET probe pairs (Lightcycler), primers incorporating a hairpin probe (Scorpion), fluorescent dyes (SYBR Green etc.), primers incorporating the complementary sequence of a DNAzyme and a cleavable fluorescent DNAzyme substrate or oligonucleotide blockers to enable the RT-PCR or an equivalent amplification technique to be carried out in real time or at end point.

In alternative embodiments, the kit further comprises, consists essentially of or consists of a gene specific reagent to allow expression of the at least one gene to be determined at the protein level. Any gene specific reagent may be employed which can specifically bind to the protein of interest. In some embodiments, the gene specific reagent comprises, consists essentially of or consists of an antibody or a derivative thereof retaining specific binding function. Antibodies and their derivatives are known in the art and discussed in more detail herein.

The invention also provides primer pairs for bisulphite genomic sequencing or methylation-specific PCR or RT-PCR. The primer pairs are selected from primers pairs comprising, consisting essentially of or consisting of the nucleotide sequences set forth in Table 1 below. Thus, in a further aspect the invention provides primer pairs for bisulphite genomic sequencing or methylation-specific PCR or RT-PCR selected from primers pairs comprising, consisting essentially of or consisting of the nucleotide sequences set forth in Table 1. The primer pairs are readily derivable from the information set forth in the table. More specifically, the invention provides bisulphite genomic sequencing primers or primers pairs comprising, consisting essentially of or consisting of the nucleotide sequences set forth as SEQ ID NOs 11-30 respectively. The invention also provides methylation-specific PCR primers or primer pairs selected from the primers or primer pairs comprising, consisting essentially of or consisting of the nucleotide sequences set forth as SEQ ID NOs 31-46. The invention also provides RT-PCR primers and primer pairs selected from the primers and primer pairs comprising, consisting essentially of or consisting of the nucleotide sequences set forth as SEQ ID NOs 47-54. Variants of these primers are also envisaged within the scope of the invention. In particular, additional flanking sequences may be added, for example to improve binding specificity, as required. Variant sequences preferably have at least 90%, at least 91%, at least 92%, at least 93%, at least 94%, at least 95%, at least 96%, at least 97%, at least 98%, or at least 99% nucleotide sequence identity with the nucleotide sequences of the primers and/or probes set forth herein. The primers and probes may incorporate synthetic nucleotide analogues as appropriate or may be DNA, RNA or PNA based for example, or mixtures thereof. Similarly alternative fluorescent donor and acceptor moieties/FRET pairs may be utilised as appropriate in real-time and end-point applications. In addition to being labelled with the fluorescent donor and acceptor moieties, the primers and probes may include modified oligonucleotides and other appending groups and labels provided that the functionality as a primer and/or probe in the methods and kits of the invention is not compromised.

TABLE 1
PRIMERS USEFUL IN THE METHODS AND KITS OF THE INVENTION

Primers for gPCR on MeDIP samples:

| | | |
|---|---|---|
| H3b Sense | CCCACACTTCTTATGCGACA | SEQ ID NO: 1 |
| H3b Antisense | CCCACACTTCTTATGCGACA | SEQ ID NO: 2 |
| OCT4 Sense | CCACTAGCCTTGACCTCTGG | SEQ ID NO: 3 |
| OCT4 Antisense | GAGCAGAAGGATTGCTTTGG | SEQ ID NO: 4 |
| H19 Sense | GAGCCGCACCAGATCTTCAG | SEQ ID NO: 5 |
| H19 Antisense | TTGGTGGAACACACTGTGATCA | SEQ ID NO: 6 |
| GPR109 Sense | CTCCTTGCTGGAGCATTCAC | SEQ ID NO: 7 |
| GPR109 Antisense | GGCAACACCTTGACAATGAA | SEQ ID NO: 8 |
| RARβ2 Sense | CCGCAAATAAAAAGGCGTAA | SEQ ID NO: 9 |
| RARβ2 Antisense | AAAGCAGACAGCCAGAGAGG | SEQ ID NO: 10 |

Primers for bisulfite genomic sequencing:

| | | |
|---|---|---|
| BID Sense | AAATAGTTTGGGGATTTTGAAT | SEQ ID NO: 11 |
| BID Antisense | AATACACTCACCACCCTCC | SEQ ID NO: 12 |
| ELAC2 Sense | GAAGGTTTTTTTTGGTATTGTG | SEQ ID NO: 13 |
| ELAC2 Antisnese | TCTCCACCAAAACTAAAAAAAC | SEQ ID NO: 14 |
| PLEKHE1 Sense | GGGAAAATAAAAGTTATTTGGT | SEQ ID NO: 15 |
| PLEKHE1 Antisense | AAACRAAACTTCAAAATAAACA | SEQ ID NO: 16 |
| DPPA4 Sense | TTGGAGAATTATTTGAGGGTAG | SEQ ID NO: 17 |
| DPPA4 Antisense | ACAACAATAAACTTCAAAAACCA | SEQ ID NO: 18 |
| UBE2V2 Sense | GTTTTGTTGTTTAGGTTGGAGTG | SEQ ID NO: 19 |
| UBE2V2 Antisense | TACTATTTCCRCAATTCCCTTA | SEQ ID NO: 20 |
| PLK2 Sense | TTTTGTTTTGTAYGTTGAGGTT | SEQ ID NO: 21 |
| PLK2 Antisense | ACACCCCRATCCACTTATAC | SEQ ID NO: 22 |
| HOXD1 Sense | GTAGAGGATTTAGAAGAGGGGA | SEQ ID NO: 23 |
| HOXD1 Antisense | ACCAAATTAACCAAAACAACC | SEQ ID NO: 24 |
| SCNN1B Sense | TGGGAAAAGTGGTTGTATATGTT | SEQ ID NO: 25 |
| SCNN1B Antisense | ACRCCAAATTCAAAAACACTA | SEQ ID NO: 26 |
| BHLHB9 Sense | GTTTGGTTAAGGAGTTTTAGGA | SEQ ID NO: 27 |
| BHLHB9 Antisense | AACATAAAAAAACACCTACCTACC | SEQ ID NO: 28 |
| RASGRF2 Sense | TTGAGTGTGTGTATTGTGGATT | SEQ ID NO: 29 |
| RASGRF2 Antisense | AAAAACCRATTTAAAAAACAAAA | SEQ ID NO: 30 |

Primers for methylation-specific PCR:

| | | |
|---|---|---|
| HOXD1 Methylated Sense | GAGTAGAAGCGGTTCGTTTC | SEQ ID NO: 31 |
| HOXD1 Methylated Antisense | ACAAAACGCCTACTCTCGA | SEQ ID NO: 32 |
| HOXD1 Unmethylated Sense | TTAGAGTAGAAGTGGTTTGTTTT | SEQ ID NO: 33 |
| HOXD1 Unmeth. Antisense | AACAAAACACCTACTCTCAAAC | SEQ ID NO: 34 |
| SCNN1B Methylated Sense | GTGTGGTTAGGTCGGTAGC | SEQ ID NO: 35 |
| SCNN1B Methylated Antisense | AACACTAAAACACCCGACG | SEQ ID NO: 36 |
| SCNN1B Unmethylated Sense | GTGTGGTTAGGTTGGTAGT | SEQ ID NO: 37 |
| SCNN1B Unmethylated Antisense | AACACTAAAACACCCAACA | SEQ ID NO: 38 |
| BHLHB9 Methylated Sense | CGTAGTTACGTGGGGTTGAC | SEQ ID NO: 39 |
| BHLHB9 Methylated Antisense | GAAACACAAATATCGTCCGC | SEQ ID NO: 40 |
| BHLHB9 Unmeth. Sense | TTGTGTAGTTATGTGGGGTTGAT | SEQ ID NO: 41 |
| BHLHB9 Unmeth. Antisense | ACAAAACACAAATATCATCCACC | SEQ ID NO: 42 |
| RASGRF2 Methylated Sense | GGTTTTCGTAATTTTGGGC | SEQ ID NO: 43 |
| RASGRF2 Methylated Antisense | AAACCGAAACACGCTCTC | SEQ ID NO: 44 |
| RASGRF2 Unmethylated Sense | GGTTTTTGTAATTTTGGGT | SEQ ID NO: 45 |
| RASGRF2 Unmethylated Antisense | AAACCAAAACACACTCTC | SEQ ID NO: 46 |

Primers for RT-PCR:

| | | |
|---|---|---|
| HOXD1 Sense | ACCTACCCCAAGTCCGTCTC | SEQ ID NO: 47 |
| HOXD1 Antisense | GTCAGTTGCTTGGTGCTGAA | SEQ ID NO: 48 |
| SCNN1B Sense | AGTGCTACCCAGGCATTGAC | SEQ ID NO: 49 |
| SCNN1B Antisense | GTCATGCCCCAGTTGAAGAT | SEQ ID NO: 50 |
| BHLHB9 Sense | ACCTTCAGCAGGTCTGCACT | SEQ ID NO: 51 |
| BHLHB9 Antisense | GGCCTTGGTTCTCAATGTGT | SEQ ID NO: 52 |
| RASGRF2 Sense | CTACTTCGAGGGCGAGCA | SEQ ID NO: 53 |
| RASGRF2 Antisense | TCCAGTGGCTTCTGACCTTC | SEQ ID NO: 54 |

### EXPERIMENTAL SECTION

### Abstract

CpG island promoter hypermethylation of tumor suppressor genes is a common hallmark of human cancer, and new large-scale epigenomic technologies might be useful in our attempts to define the complete DNA hypermethylome of tumor cells. Here we report a functional search for hypermethylated CpG islands using the colorectal cancer cell line HCT-116, in which two major DNA methyltransferases, DNMT1 and DNMT3b, have been genetically disrupted (DKO cells). Using methylated DNA immunoprecipitation (MeDIP) methodology in conjunction with promoter microarray analyses we found that DKO cells experience a significant loss of hypermethylated CpG islands. Further characterization of these candidate sequences demonstrates CpG island promoter hypermethylation and silencing of genes with potentially important roles in tumorigenesis, such as the Ras guanine-nucleotide release factor RASGRF2, the apoptosis-associated basic helixloop transcription factor BHLHB9, and the homeobox gene HOXD1. Hypermethylation of these genes occurs already in premalignant lesions and accumulates during tumorigenesis. Thus, our results demonstrate the usefulness of DNMT genetic disruption strategies combined with MeDIP in searching for unknown hypermethylated candidate genes in human cancer that might aid our understanding of the biology of the disease and be of potential translational use.

### Materials and Methods

### Human Cancer Cell Lines and Primary Tumor Samples.

HCT-116 colon cancer cells and double DNMT1−/−DNMT3b−/− (DKO) cells were grown as previously described (5). HCT-116 cells were treated with 5-aza-2-deoxycytidine (1 μmol/L) for 72 h. HCT116 and DKO cells were a generous gift from Dr. Bert Vogelstein (Johns Hopkins Kimmel Comprehensive Cancer Center, Baltimore, Md.). All the other human colon cancer cell lines (n=7) were obtained from the American Type Culture Collection (Rockville, Md.). Tissue samples of primary colorectal tumors (n=72), adenomas (n=34) and normal colon (n=10) were all obtained at the time of the clinically indicated procedures.

### Methylated DNA Immunoprecipitation (MeDIP) Assay.

The MeDIP assay was developed as previously described (8). 4 μg of genomic DNA extracted from HCT116 and DKO nuclei were sonicated to produce random fragments ranging in size from 300 to 600 bp. We denatured the DNA for 10 min at 95° C. and immunoprecipitated it overnight at 4° C. with 10 μL of monoclonal antibody against 5-methylcytidine (Eurogentec) in a final volume of 500 μL immunoprecipitation buffer (10 mM sodium phosphate, pH 7.0, 140 mM NaCl, 0.05% Triton X-100). We incubated the mixture with 30 μl of Dynabeads with M-280 sheep antibody to mouse IgG (Dynal Biotech) for 2 h at 4° C. and washed it three times with 700 μL of IP buffer. We then treated the beads with proteinase K for 3 h at 50° C. and recovered the methylated DNA by phenol-chloroform extraction followed by ethanol precipitation.

### Real-Time PCR on MeDIP Samples.

We carried out real-time PCR reactions with 10 ng of input DNA and ¼ of the immunoprecipitated methylated DNA. For realtime PCR reactions, we used the SYBR Green PCR master mix (Applied Biosystems, Foster City, Calif.) and a 7900HT Fast Real-Time PCR system (Applied Biosystems, Foster City, Calif.). Reactions were done in triplicate and standard curves were calculated on serial dilutions (100-0.1 ng) of input genomic DNA. To evaluate the relative enrichment of target sequences after MeDIP, we calculated the ratios of the signals in the immunoprecipitated DNA with respect to input DNA. The resulting values were compared with an unmethylated control gene, histone H3.

### MeDIP-on-ChIP.

The enriched DNA obtained from the MeDIP assays was labeled and purified using the Invitrogen Bioprime random primer labeling kit (immunoprecipitated methylated DNA was labeled with cy5 fluorophere and the Input genomic DNA was labeled with Cy3 fluorophere). Labeled DNA from the enriched and the input pools were combined (1-2 μg) and hybridized to the Human Proximal Promoter Array 44K (Agilent Technologies, Palo Alto, Calif.), according to the manufacturer's protocols. Arrays were then washed and scanned with an Agilent DNA microarray scanner.

### Data Normalization and Analysis.

The microarray data were extracted with Feature Extraction Software v9.1 (Agilent Technologies, Palo Alto, Calif.). ChIP analytics 1.2 (Agilent Technologies, Palo Alto, Calif.) was used to normalize the data (Blanks Subtraction Normalization; Inter-array Median Normalization; Intra-array Median Normalization) and to determine bound regions in the datasets, using an algorithm that incorporates information from neighboring probes. Probe sets were marked as potentially bound if the p-value of average X (probe set p-values) was less than 0.001. They were also required to be selected by one of two additional filters: a) two of the three probes in a probe set should have single probe p-values<0.05 or the central probe in the set should have a single probe p-value<0.001 and b) one of the flanking probes should have a single point p-value<0.1. To obtain more information about the biological features of the hypermethylated candidate genes and to check the biological coherence of the results obtained, we used the FatiGO program (11). This identifies Gene Ontology (GO) terms for biological processes or molecular functions of genes that are over- or underrepresented, using the total number of genes annotated to a particular GO term as a reference.

### DNA Methylation Analysis of Candidate Genes.

The CpG Island Searcher Program (12) was used to determine which genes had a CpG island in their 5′-ends. DNA methylation status was established by PCR analysis of bisulfite-modified genomic DNA. This induces chemical conversion of unmethylated, but not methylated, cytosine to uracil, using two procedures as previously described. First, methylation status was analyzed by bisulfite genomic sequencing of both strands of the corresponding CpG islands. The second procedure used methylation-specific PCR involving primers specific to either themethylated or modified unmethylated DNA. The primers used are described in Supplementary Table S1.

### Semiquantitative RT-PCR Expression Analysis.

We reverse-transcribed total RNA (2 μg) treated with Dnase I (Ambion) using oligo (dT) 12-18 primer with Superscript II reverse transcriptase (Gibco BRL). We carried out PCR reactions in a 50 μl volume containing 1×PCR buffer (Gibco BRL), 1.5 mM of MgCl2, 0.3 mM of dNTP, 0.25 μM of each primer and 2 U of Taq polymerase (Gibco BRL). We used 100 ng of cDNA for PCR amplification, and we amplified all of the genes with multiple cycle numbers (20-35 cycles) to determine the appropriate conditions for obtaining semiquantitative differences in their expression levels. RT-PCR primers were designed between different exons to avoid any amplification of DNA. We also carried out PCR with GAPDH (25 and 28 cycles) to ensure cDNA quality and loading accuracy. The primers used are described in Supplementary Table S1.

### Results and Discussion

### The MeDIP Microarray Profile of Wild-Type HCT-116 Colon Cancer Cells and DNA Methyltransferase-Deficient Cells (DKO).

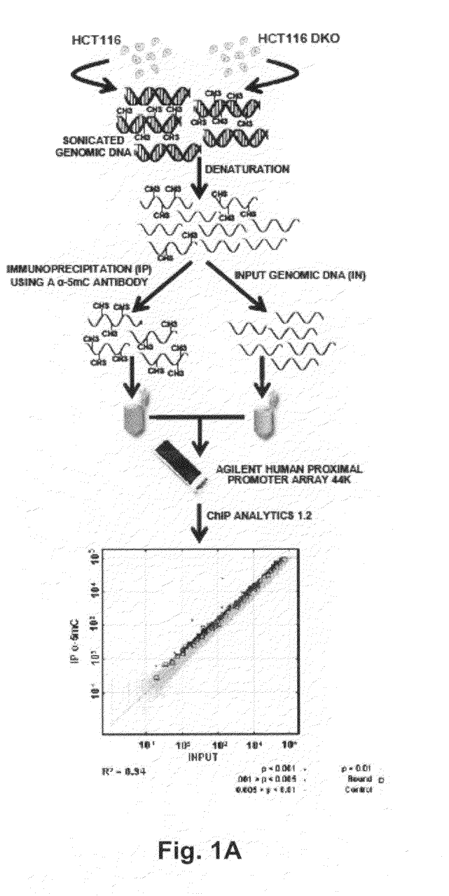

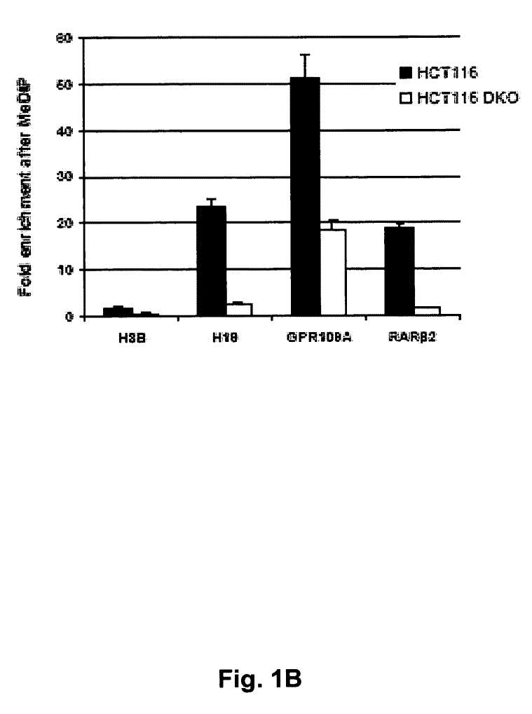

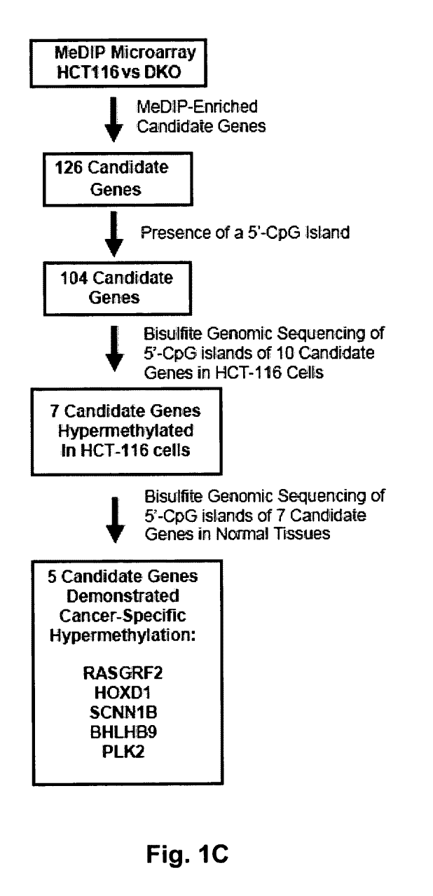

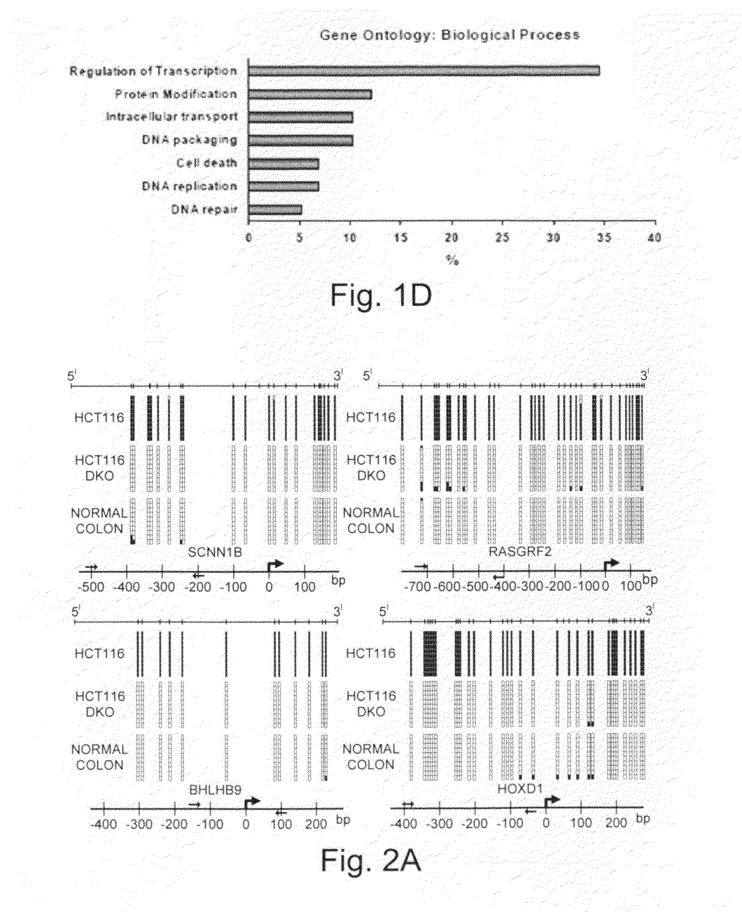

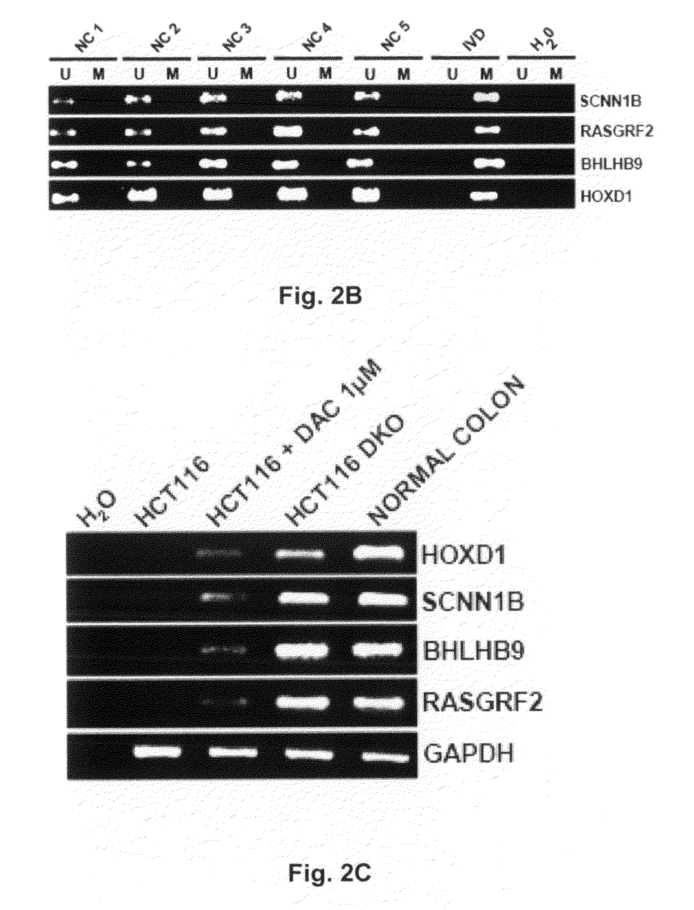

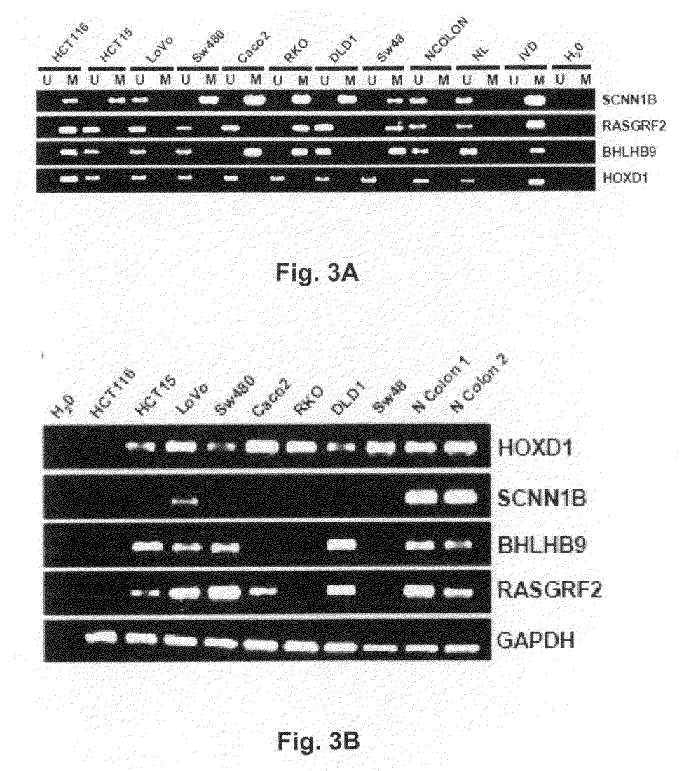

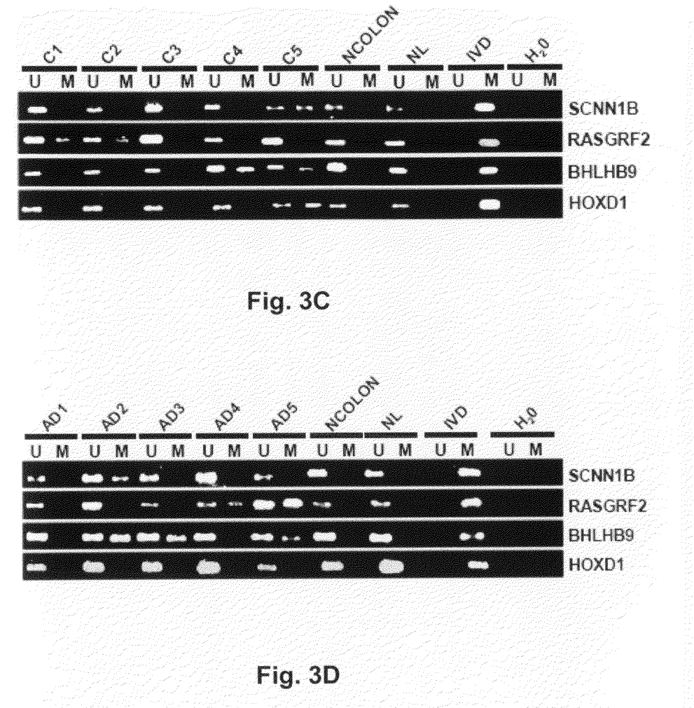

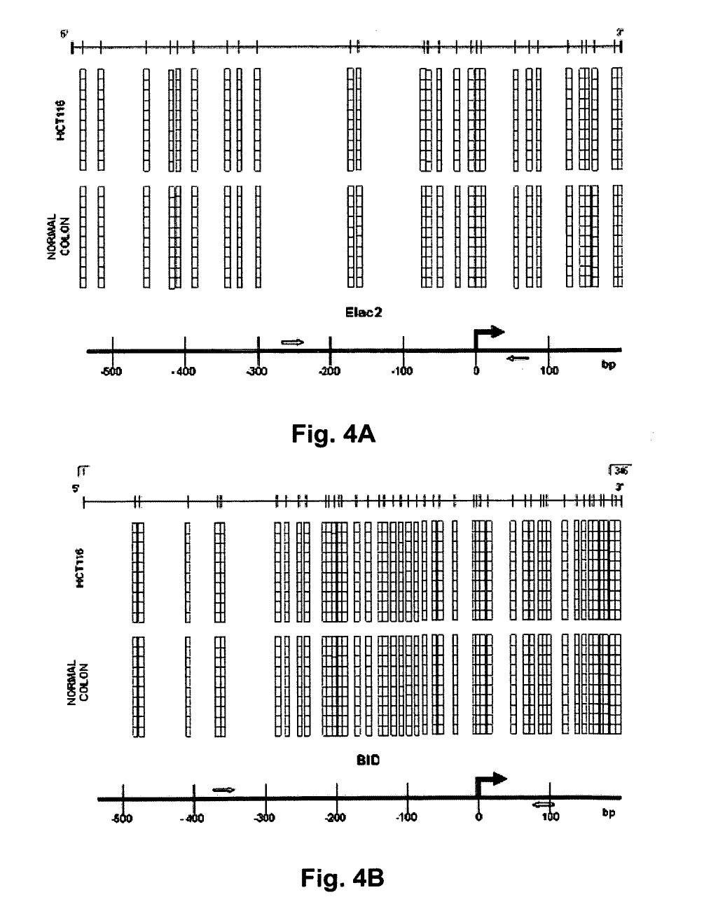

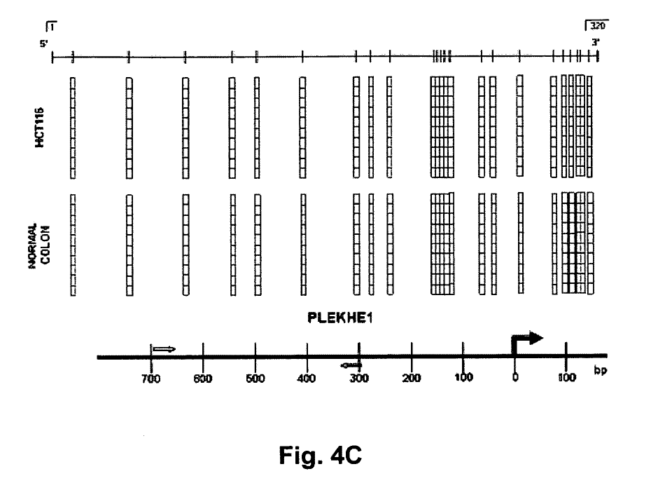

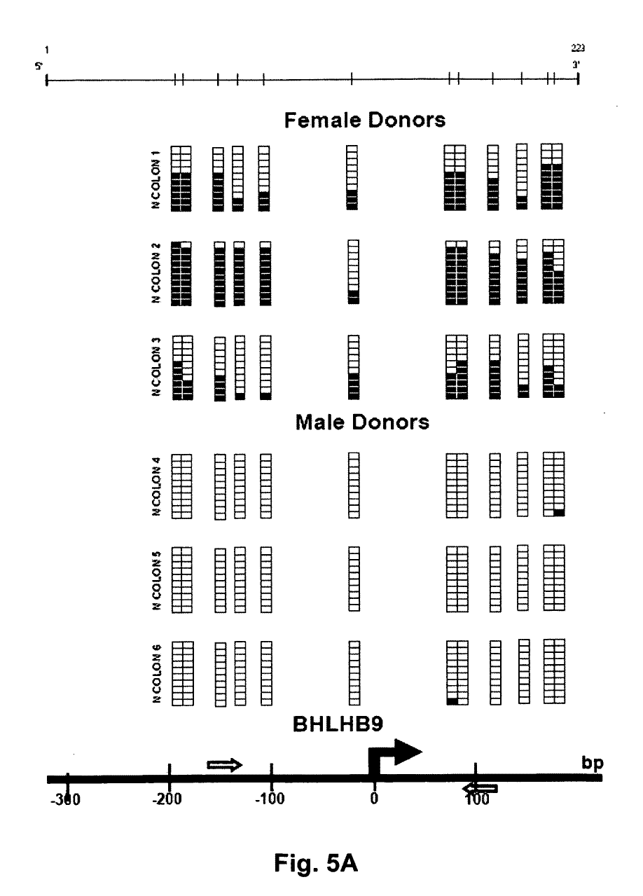

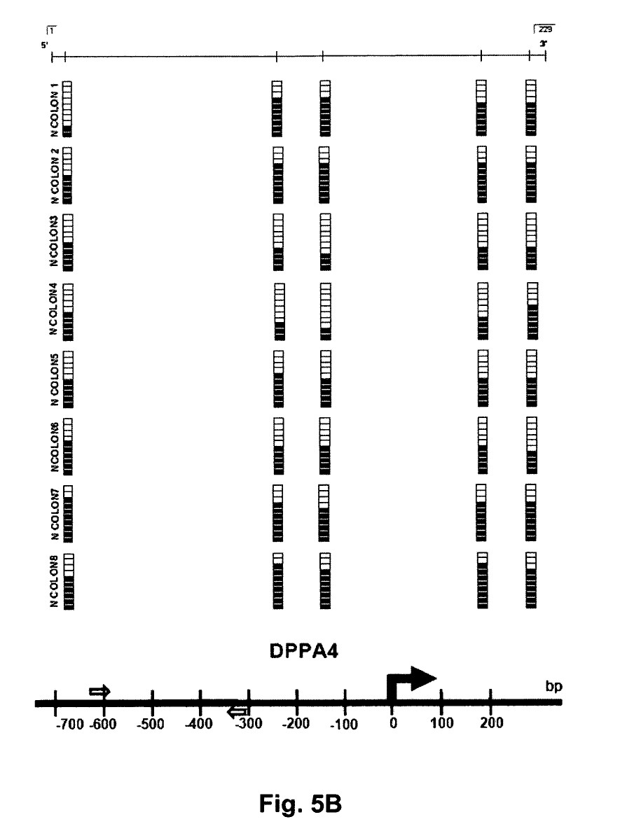

MeDIP has been used in conjunction with genomic microarrays in transformed and normal cells to outline the DNA methylation differences associated with tumorigenesis in several recent, promising studies (8-10). We have applied the MeDIP approach to a 44K human proximal promoter array to evaluate the CpG hypomethylation changes in the DNMT1/DNMT3b double knockout HCT-116 cells (DKO) in relation to the wild-type HCT-116 to reveal newly hypermethylated genes in colorectal tumors. The methodology is summarized in FIG. 1A. To test the specificity and efficiency of MeDIP, we compared the relative enrichment of known methylated and unmethylated genomic sequences using real-time PCR. MeDIP enriched methylated DNA, as exemplified by the cancer-specific promoter hypermethylation of the Retinoic Acid Receptor B2 (RARB2) (1-3) and the imprinting control regions of H19 and GPR109A (8, 13) (FIG. 1B), in comparison with unmethylated CpG sequences, such as the Histone H3B promoter (10) (FIG. 1B). Most importantly, DKO cells showed markedly depleted MeDIP enrichment in comparison with HCT-116 cells for the methylated DNA sequences, such as RARB2, H19, and GPR109A (FIG. 1B).

From the global genomic perspective, we observed abundant DNA demethylation events in DKO cells in comparison to wild-type HCT-116 cells. Of the 44,000 printed promoters in the array, we observed significant 5-methylcytosine DNA immunoprecipitation losses in 126 candidate genes in the DKO cells (FIG. 1C). Using the CpG Island Searcher Program (11) we estimated that 104 (83%) of these candidate genes had a CpG island in their 5′-ends (FIG. 1C). Gene ontology analyses of these 104 candidate hypermethylated genes revealed a broad representation of all the common hallmark pathways of cancer cells (14), such as DNA repair, and cell death, although transcriptional regulators were clearly over-represented (FIG. 1D). This latter observation is of particular interest since many hypermethylated promoter CpG islands in cancer cells correspond to genes with a critical role in the regulation of transcription, such as SFRPs-1, GATAs, HIC-1, DKK-1 and TWIST2 (1-3, 15).

To gain further knowledge of the different DNA methylation patterns of the genes enriched after MeDIP in HCT-116 and DKO cells, we randomly selected ten of these gene-associated-CpG islands for further characterization by bisulfite genomic sequencing of multiple clones. The genes selected were the Ras protein-specific guanine nucleotide-releasing factor 2 (RASGRF2), the Sodium channel nonvoltage-gated 1 beta (SCNN1B), the homeobox D1 (HOXD1), the Polo-like kinase 2 (PLK2), the Basic helixloop-helix domain containing class B9 (BHLHB9), the Developmental pluripotency associated 4 (DPPA4), Ubiquitin-conjugating enzyme E2 variant 2 (UBE2V2), the ElaC homolog 2 (ELAC2), the BH3 interacting domain death agonist (BID) and the PH domain and leucine rich repeat protein phosphatase 1 (PLEKHE1). We observed that 70% (7/10) of these CpG islands were densely methylated in HCT-116 cells and fully unmethylated in DKO cells (FIG. 1C and FIG. 2A). The genes were RASGRF2, SCNN1B, HOXD1, PLK2, BHLHB9, DPPA4 and UBE2V2. In the three remaining genes, ELAC2, BID, and PLEKHE1, the 5′-associated CpG islands were unmethylated in both HCT-116 and DKO cells (Supplementary Fig. S1). We wanted to focus on cancerspecific DNA hypermethylation, and so we analyzed the DNA methylation status of the seven CpG islands found to be hypermethylated in HCT-116 cells by bisulfite genomic sequencing in a collection of normal colon tissues (n=10). We observed that 57% (4/7) of these CpG islands were always unmethylated in the normal tissues, and thus their methylation was cancer-specific: this was the case for RASGRF2, SCNN1B, HOXD1, and PLK2 (FIGS. 2A and 2B).

For the three remaining genes, the CpG islands of DPPA4 and UBE2V2 were consistently methylated in all normal colon samples studied (data not shown), but the case of the third gene, BHLHB9, was particularly interesting due to its chromosomal location on the X-chromosome (Xq23), that, in females, it is randomly inactivated by DNA methylation in one copy. Thus, the normal tissues in which we found BHLHB9 CpG island hypermethylation were all from female donors whilst normal tissues from male donors were always unmethylated (Supplementary Figure S2). Most importantly, because the HCT-116 cancer cells originated from a male (16), and thus had not undergone Xinactivation and its associated BHLHB9 CpG island hypermethylation, the presence of BHLHB9 hypermethylation in the HCT-116 malignant cells can be considered a cancerspecific hypermethylation event, similar to those described for the other four newly identified candidates (RASGRF2, SCNN1B, HOXD1, and PLK2).

CpG Island Hypermethylation in the Newly Identified Candidate Genes is Associated with Transcriptional Inactivation.

It is critical to establish the impact of the detected 5′-end CpG island hypermethylation events on the expression of the contiguous genes. The presence of PLK2 hypermethylation-associated silencing has recently been described (17) and for this reason we undertook no further studies. For the four remaining hypermethylated cancer-specific genes (RASGRF2, SCNN1B, HOXD1, and BHLHB9), we addressed the association between DNA methylation and expression analyzed by semiquantitative RT-PCR. For all four genes, hypermethylated HCT-116 cells showed loss of expression of their respective transcripts (FIG. 2C). Most importantly, restoration of expression was observed upon treatment with the demethylating agent 5-aza-2-deoxycytidine and was also observed in DKO cells (FIG. 2C), further strengthening the evidence for the role of 5′-CpG island hypermethylation in their transcriptional silencing.

The presence of hypermethylation of RASGRF2, SCNN1B, HOXD1, and BHLHB9 was not a unique feature of the HCT-116 colon cancer cell line; upon analyzing a collection of colorectal cancer cell lines (n=22), we commonly observed the presence of these epigenetic alteration (FIG. 3A and Table 2). The only exception was HOXD1 that was only hypermethylated in HCT-116. Because all cell lines used were originated from male colorectal patients, except SW48, the normal X-chromosome related hypermethylation of BHLHB9 in females was not an issue. The association between CpG island hypermethylation of each gene and loss of expression, demonstrated above in HCT-116 cells, was also found in this panel of cancer cell lines (FIG. 3B).

### CpG Island Hypermethylation Profile for the Newly Identified Candidate Genes in Human Colorectal Tumorigenesis.

The presence of RASGRF2, SCNN1B, HOXD1, and BHLHB9 hypermethylation was not an in vitro cell culture phenomenon, but when a collection of primary tumor samples from colorectal cancer patients was analyzed, it was also commonly observed (FIG. 3C and Table 2). The presence of hypermethylation of any of the described four genes was not associated with any particular clinical stage, age of the patient, or anatomical location in the colon.

To determine whether hypermethylation of the described genes might represent an early lesion in colorectal tumorigenesis, we examined the CpG island methylation status of RASGRF2, SCNN1B, HOXD1, and BHLHB9 in benign colorectal adenomas, a lesion that is a precursor to invasive colorectal tumors. We observed a hypermethylation frequency similar to that of invasive colon carcinomas (FIG. 3C and Table 2), it being present in both small (<15 mm) and large adenomas (>15 mm). These findings point to hypermethylation of the identified candidate genes as early events in the pathway to full blown colorectal tumors.

Our results suggest that the genomic disruption of the DNMTs associated with a MeDIP-promoter microarray approach is a useful strategy for “catching” new genes undergoing DNA methylation-associated silencing in human cancer. The generation of cancer cells whose two major DNA methyltransferases are disrupted provides a pure population of cancer cells that can be compared to the original cell line because they only differ with respect to their DNA methylation pattern. By using such DKO cells we had previously revealed a constellation of genes with methylation-associated silencing in human tumors using global DNA methylation techniques (7). Now, we have gone one step further by comparing HCT-116 wild-type cells and DKO cells using the newly developed MeDIP approach (8-10) in association with a comprehensive promoter microarray platform. We have shown that the CpG island hypermethylation of these newly identified genes is not a unique feature of HCT-116 cells, but is also common among colorectal tumorigenesis, it being found in other colon cancer cell lines, primary colon tumors, and colon adenomas.

The list of epigenetically silenced genes revealed covers most of the disrupted pathways of cancer cells (14), such as ras-mediated signal transduction and development, exemplified by RASGRF2 and HOXD1, respectively. These two cases are not isolated epigenetic events in their categories, but are added to other ras-related genes such as the ras effectors RASSF1A (18) and NORE1A (19), and homeobox genes such as HOXA9, LMX-1, HOXA5 and DUX-4 (1-3, 7) undergoing methylation-associated silencing in human cancer cells. SCNN1B is another interesting case because it codes for the beta subunit of an epithelial sodium channel and transcriptional silencing by CpG island hypermethylation of other ion channels such as CACNA1G (20) and CALCA1l (7) seems to be a common finding in human tumors. Furthermore, CALCA1l transfection in colon cancer cells provokes a marked reduction of colony formation (7). These data, and the newly identified epigenetic silencing of SCNN1B in our experiments, are evidence in favor of the proposed role of sodium, calcium, potassium, and chloride channels in the regulation of cell proliferation, migration, and invasion (21). Finally, the identification of CpG island hypermethylation of the bHLHB9 gene pinpoints another family of proteins critical to tumorigenesis, the basic helix-loop (bHLH) factors (22). The bHLH family of transcription factors functions in the coordinated regulation of gene expression, cell lineage commitment, and cell differentiation in most tissues (22). In the case of bHLHB9, a pivotal role in apoptotic cell death has been proposed (23). Interestingly, in a similar manner to which it occurs in the ion channels, bHLHB9 hypermethylation is not an isolated event in its category; other bHLH proteins, such as TWIST2 (15), also undergo methylation-associated silencing in human neoplasias. This further underlines the role of this family of transcription factors in cellular transformation.

Thus, overall, we have demonstrated that the use of powerful epigenomic technologies, such as MeDIP in conjunction with comprehensive promoter microarrays, in cancer cells whose DNMT genes have been disrupted, can identify new hypermethylated genes in human colorectal tumorigenesis. These aberrantly epigenetically silenced genes are members of the various cellular pathways that contribute to the tumorigenic phenotype and illustrate the disrupted DNA methylation landscape present in cancer cells.

TABLE 2
CpG island hypermethylation distribution
of the MeDIP-identified candidate genes

| | Colorectal | Colorectal | | | | | |
|---|---|---|---|---|---|---|---|
| | Cancer Cell | Primary | | | | Normal | |
| Genes | Lines | Tumours | | Adenomas | | Colon | |
| SCNN1B | 88% (7/8) | 20% | (11/56) | 13% | (4/30) | 0% | (0/10) |
| RASGRF2 | 38% (3/8) | 45% | (29/65) | 35% | (12/34) | 0% | (0/10) |
| BHLHB9* | 42% (3/7) | 33% | (18/54) | 33% | (9/27) | 0% | (0/7) |
| HOXD1 | 13% (1/8) | 4% | (2/52) | 0% | (0/33) | 0% | (0/10) |

*For BHLHB9, located in the X-chromosome, only samples from male donors are included

### REFERENCES

1. Jones P A, Laird P W. Cancer epigenetics comes of age. Nat Genet 1999; 21:163-7.
2. Herman J G, Baylin S B. Gene silencing in cancer in association with promoter hypermethylation. N Engl J Med 2003; 349:2042-54.
3. Esteller M. Cancer epigenomics: DNA methylomes and histone-modification maps. Nat Rev Genet 2007; 8:286-98.
4. Rhee I, Jair K W, Yen R W, Lengauer C, Herman J G, Kinzler K W, et al. CpG methylation is maintained in human cancer cells lacking DNMT1 Nature 2000; 404:1003-7.
5. Rhee I, Bachman K E, Park B H, Jair K W, Yen R W, Schuebel K E, et al. DNMT1 and DNMT3b cooperate to silence genes in human cancer cells. Nature 2002; 416:52-6.
6. Egger G, Jeong S, Escobar S G, Cortez C C, Li T W, Saito Y, et al. Identification of DNMT1 (DNA methyltransferase 1) hypomorphs in somatic knockouts suggests an essential role for DNMT1 in cell survival. Proc Natl Acad Sci USA 2006; 103:14080-5.
7. Paz M F, Wei S, Cigudosa J C, Rodriguez-Perales S, Peinado M A, Huang T H, et al. Genetic unmasking of epigenetically silenced tumor suppressor genes in colon cancer cells deficient in DNA methyltransferases. Hum Mol Genet 2003; 12:2209-19.
8. Weber M, Davies J J, Wittig D, Oakeley E J, Haase M, Lam W L, et al. Chromosome-wide and promoter-specific analyses identify sites of differential DNA methylation in normal and transformed human cells. Nat Genet 2005; 37:853-62.
9. Keshet I, Schlesinger Y, Farkash S, Rand E, Hecht M, Segal E, et al. Evidence for an instructive mechanism of de novo methylation in cancer cells. Nat Genet 2006; 38:149-53.
10. Weber M, Hellmann I, Stadler M B, Ramos L, Paabo S, Rebhan M, et al. Distribution, silencing potential and evolutionary impact of promoter DNA methylation in the human genome. Nat Genet 2007; 39:457-66.
11. Takai D, Jones P A. The CpG island searcher: a new WWW resource. In Silico Biol 2003; 3:235-40.
12. Al-Shahrour, Diaz-Uriarte R, Dopazo J. FatiGO: a web tool for finding significant associations of Gene Ontology terms with groups of genes. Bioinformatics 2004; 20:578-80.
13. Feinberg A P. Imprinting of a genomic domain of 11p15 and loss of imprinting in cancer: an introduction. Cancer Res 1999; 59(7 Suppl):1743s-6s.
14. Hanahan D, Weinberg R A. The hallmarks of cancer. Cell 2000; 100:57-70.
15. Raval A, Lucas D M, Matkovic J J, Bennett K L, Liyanarachchi S, Young D C, et al. TWIST2 demonstrates differential methylation in immunoglobulin variable heavy chain mutated and unmutated chronic lymphocytic leukemia. J Clin Oncol 2005; 23:3877-85.
16. Brattain M G, Fine W D, Khaled F M, Thompson J, Brattain D E. Heterogeneity of malignant cells from a human colonic carcinoma. Cancer Res 1981; 41:1751-6.
17. Syed N, Smith P, Sullivan A, Spender L C, Dyer M, Karran L, et al. Transcriptional silencing of Polo-like kinase 2 (SNK/PLK2) is a frequent event in Bcell malignancies. Blood 2006; 107:250-6.
18. Dammann R, Li C, Yoon J H, Chin P L, Bates S, Pfeifer G P. Epigenetic inactivation of a RAS association domain family protein from the lung tumour suppressor locus 3p21.3. Nat Genet 2000; 25:315-9.
19. Hesson L, Dallol A, Minna J D, Maher E R, Latif F. NORE1A, a homologue of RASSF1A tumour suppressor gene is inactivated in human cancers. Oncogene 2003; 22:947-54.
20. Toyota M, Ho C, Ohe-Toyota M, Baylin S B, Issa J P. Inactivation of CACNA1G, a T-type calcium channel gene, by aberrant methylation of its 5′ CpG island in human tumors. Cancer Res 1999; 59:4535-41.
21. Kunzelmann K. Ion channels and cancer. J Membr Biol 2005; 205:159-73.
22. Desprez P Y, Sumida T, Coppe J P. Helix-loop-helix proteins in mammary gland development and breast cancer. J Mammary Gland Biol Neoplasia 2003; 8:225-39.
23. Heese K, Yamada T, Akatsu H, Yamamoto T, Kosaka K, Nagai Y, Sawada T. Characterizing the new transcription regulator protein p60TRP. J Cell Biochem. 2004 April 1 ; 91(5):1030-42.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

Various publications are cited herein, the disclosures of which are incorporated by reference in their entireties.

1. A method of identifying nucleic acid molecules differentially methylated in a disease comprising:
(a) incubating fragmented DNA, from a disease cell, with a reagent which specifically binds to methylated DNA to thus concentrate methylated DNA fragments
(b) incubating fragmented DNA, from a disease cell related to the disease cell utilised in step (a) in which DNA methyltransferase expression and/or activity has been inhibited, with a reagent which specifically binds to methylated DNA to thus concentrate methylated DNA fragments
(c) comparing the methylated DNA fragments obtained in steps (a) and (b) to identify nucleic acid molecules differentially methylated in the disease.

2. The method of claim 1 wherein step (c) comprises differentially labelling the methylated DNA fragments obtained in steps (a) and (b) and hybridizing the methylated DNA fragments to a microarray to identify nucleic acid molecules differentially methylated in the disease

3. The method of claim 1 wherein nucleic acid molecules differentially methylated in the disease are further characterised by determining the presence or absence of a CpG island in the nucleotide sequence.

4. The method of claim 3 further comprising determining the methylation status of the CpG island of the nucleic acid molecules which include a CpG island from a disease cell to identify nucleic acid molecules which are methylated in the disease cell.

5. The method of claim 4 further comprising determining the methylation status of the CpG island of the nucleic acid molecules which include a CpG island in a non-disease cell wherein a lack of methylation or a lesser degree of methylation in the non-disease cell indicates that the nucleic acid molecule is methylated as an indicator of the disease.

6. The method of claim 1 further comprising determining the effect of methylation on expression of the nucleic acid molecule by comparing gene expression in the disease cell and disease cell in which DNA methyltransferase expression and/or activity has been inhibited.

7. The method of any of claim 6 further comprising determining whether use of a demethylating agent can restore expression of the nucleic acid molecule in the disease cell.

8. The method of claim 1 which is utilised to identify candidate tumour suppressor genes.

9. A method of detecting a predisposition to, or the incidence of, colorectal cancer in a sample comprising detecting an epigenetic change in at least one gene selected from RASGRF2, SCNN1B, HOXD1, PLK2 and BHLHB9 wherein detection of the epigenetic change is indicative of a predisposition to, or the incidence of, colorectal cancer.

10. The method of any of claim 9 wherein the epigenetic change is methylation.

11. A method for predicting the likelihood of successful treatment of colorectal cancer with a DNA demethylating agent and/or a DNA methyltransferase inhibitor and/or HDAC inhibitor comprising detecting an epigenetic change in at least one gene selected from RASGRF2, SCNN1B, HOXD1, PLK2 and BHLHB9 in a sample, wherein detection of the epigenetic change is indicative that the likelihood of successful treatment is higher than if the epigenetic modification is not detected.

12. A method for predicting the likelihood of resistance to treatment of colorectal cancer with a DNA demethylating agent and/or DNA methyltransferase inhibitor and/or HDAC inhibitor comprising detecting an epigenetic change in at least one gene selected from RASGRF2, SCNN1B, HOXD1, PLK2 and BHLHB9 in a sample, wherein detection of the epigenetic change is indicative that the likelihood of resistance to treatment is lower than if the epigenetic modification is not detected.

13. A method of selecting a suitable treatment regimen for colorectal cancer comprising detecting an epigenetic change in at least one gene selected from RASGRF2, SCNN1B, HOXD1, PLK2 and BHLHB9 in a sample, wherein detection of the epigenetic change results in selection of a DNA demethylating agent and/or a DNA methyltransferase inhibitor and/or a HDAC inhibitor for treatment and wherein if the epigenetic change is not detected, a DNA demethylating agent and/or a DNA methyltransferase inhibitor and/or a HDAC inhibitor is not selected for treatment.

14. A method of treating colorectal cancer in a subject comprising administration of a DNA demethylating agent and/or a DNA demethylating agent and/or a DNA methyltransferase inhibitor wherein the subject has been selected for treatment on the basis of a method as claimed in claim 11 or 13.

15. A kit for detecting a predisposition to, or the incidence of, colorectal cancer in a sample comprising, consisting essentially of or consisting of means for detecting an epigenetic change in at least one gene selected from RASGRF2, SCNN1B, HOXD1, PLK2 and BHLHB9.

16. The kit of claim 15 wherein the means for detecting methylation comprises, consists essentially of or consists of methylation specific PCR primers.

17. The kit of claim 15 further comprising a reagent which selectively modifies unmethylated cytosine residues in the DNA contained in the sample to produce detectable modified residues but which does not modify methylated cytosine residues.

18. A bisulphite genomic sequencing primer or primers pair selected from the primers or primer pairs comprising the nucleotide sequences set forth as SEQ ID NOs 11-30.

19. A methylation-specific PCR primer or primer pair selected from the primers or primer pairs comprising the nucleotide sequences set forth as SEQ ID NOs 31-46.

20. An RT-PCR primer or primer pair selected from the primers and primer pairs comprising the nucleotide sequences set forth as SEQ ID NOs 47-54.


SEQUENCE LISTING (54 sequences)

SEQ ID NO: 1 - DNA, 20 bp, Artificial sequence
  Primer
cccacacttcttatgcgaca

SEQ ID NO: 2 - DNA, 20 bp, Artificial sequence
  Primer
cccacacttcttatgcgaca

SEQ ID NO: 3 - DNA, 20 bp, Artificial sequence
  Primer
ccactagccttgacctctgg

SEQ ID NO: 4 - DNA, 20 bp, Artificial sequence
  Primer
gagcagaaggattgctttgg

SEQ ID NO: 5 - DNA, 20 bp, Artificial sequence
  Primer
gagccgcaccagatcttcag

SEQ ID NO: 6 - DNA, 22 bp, Artificial sequence
  Primer
ttggtggaacacactgtgatca

SEQ ID NO: 7 - DNA, 20 bp, Artificial sequence
  Primer
ctccttgctggagcattcac

SEQ ID NO: 8 - DNA, 20 bp, Artificial sequence
  Primer
ggcaacaccttgacaatgaa

SEQ ID NO: 9 - DNA, 20 bp, Artificial sequence
  Primer
ccgcaaataaaaaggcgtaa

SEQ ID NO: 10 - DNA, 20 bp, Artificial sequence
  Primer
aaagcagacagccagagagg

SEQ ID NO: 11 - DNA, 22 bp, Artificial sequence
  Primer
aaatagtttggggattttgaat

SEQ ID NO: 12 - DNA, 19 bp, Artificial sequence
  Primer
aatacactcaccaccctcc

SEQ ID NO: 13 - DNA, 22 bp, Artificial sequence
  Primer
gaaggttttttttggtattgtg

SEQ ID NO: 14 - DNA, 22 bp, Artificial sequence
  Primer
tctccaccaaaactaaaaaaac

SEQ ID NO: 15 - DNA, 22 bp, Artificial sequence
  Primer
gggaaaataaaagttatttggt

SEQ ID NO: 16 - DNA, 22 bp, Artificial sequence
  Primer
aaacraaacttcaaaataaaca

SEQ ID NO: 17 - DNA, 22 bp, Artificial sequence
  Primer
ttggagaattatttgagggtag

SEQ ID NO: 18 - DNA, 23 bp, Artificial sequence
  Primer
acaacaataaacttcaaaaacca

SEQ ID NO: 19 - DNA, 23 bp, Artificial sequence
  Primer
gttttgttgtttaggttggagtg

SEQ ID NO: 20 - DNA, 22 bp, Artificial sequence
  Primer
tactatttccrcaattccctta

SEQ ID NO: 21 - DNA, 22 bp, Artificial sequence
  Primer
ttttgttttgtaygttgaggtt

SEQ ID NO: 22 - DNA, 20 bp, Artificial sequence
  Primer
acaccccratccacttatac

SEQ ID NO: 23 - DNA, 22 bp, Artificial sequence
  Primer
gtagaggatttagaagagggga

SEQ ID NO: 24 - DNA, 21 bp, Artificial sequence
  Primer
accaaattaaccaaaacaacc

SEQ ID NO: 25 - DNA, 23 bp, Artificial sequence
  Primer
tgggaaaagtggttgtatatgtt

SEQ ID NO: 26 - DNA, 21 bp, Artificial sequence
  Primer
acrccaaattcaaaaacacta

SEQ ID NO: 27 - DNA, 22 bp, Artificial sequence
  Primer
gtttggttaaggagttttagga

SEQ ID NO: 28 - DNA, 24 bp, Artificial sequence
  Primer
aacataaaaaaacacctacctacc

SEQ ID NO: 29 - DNA, 22 bp, Artificial sequence
  Primer
ttgagtgtgtgtattgtggatt

SEQ ID NO: 30 - DNA, 23 bp, Artificial sequence
  Primer
aaaaaccratttaaaaaacaaaa

SEQ ID NO: 31 - DNA, 20 bp, Artificial sequence
  Primer
gagtagaagcggttcgtttc

SEQ ID NO: 32 - DNA, 19 bp, Artificial sequence
  Primer
acaaaacgcctactctcga

SEQ ID NO: 33 - DNA, 23 bp, Artificial sequence
  Primer
ttagagtagaagtggtttgtttt

SEQ ID NO: 34 - DNA, 22 bp, Artificial sequence
  Primer
aacaaaacacctactctcaaac

SEQ ID NO: 35 - DNA, 19 bp, Artificial sequence
  Primer
gtgtggttaggtcggtagc

SEQ ID NO: 36 - DNA, 19 bp, Artificial sequence
  Primer
aacactaaaacacccgacg

SEQ ID NO: 37 - DNA, 19 bp, Artificial sequence
  Primer
gtgtggttaggttggtagt

SEQ ID NO: 38 - DNA, 19 bp, Artificial sequence
  Primer
aacactaaaacacccaaca

SEQ ID NO: 39 - DNA, 20 bp, Artificial sequence
  Primer
cgtagttacgtggggttgac

SEQ ID NO: 40 - DNA, 20 bp, Artificial sequence
  Primer
gaaacacaaatatcgtccgc

SEQ ID NO: 41 - DNA, 23 bp, Artificial sequence
  Primer
ttgtgtagttatgtggggttgat

SEQ ID NO: 42 - DNA, 23 bp, Artificial sequence
  Primer
acaaaacacaaatatcatccacc

SEQ ID NO: 43 - DNA, 19 bp, Artificial sequence
  Primer
ggttttcgtaattttgggc

SEQ ID NO: 44 - DNA, 18 bp, Artificial sequence
  Primer
aaaccgaaacacgctctc

SEQ ID NO: 45 - DNA, 19 bp, Artificial sequence
  Primer
ggtttttgtaattttgggt

SEQ ID NO: 46 - DNA, 18 bp, Artificial sequence
  Primer
aaaccaaaacacactctc

SEQ ID NO: 47 - DNA, 20 bp, Artificial sequence
  Primer
acctaccccaagtccgtctc

SEQ ID NO: 48 - DNA, 20 bp, Artificial sequence
  Primer
gtcagttgcttggtgctgaa

SEQ ID NO: 49 - DNA, 20 bp, Artificial sequence
  Primer
agtgctacccaggcattgac

SEQ ID NO: 50 - DNA, 20 bp, Artificial sequence
  Primer
gtcatgccccagttgaagat

SEQ ID NO: 51 - DNA, 20 bp, Artificial sequence
  Primer
accttcagcaggtctgcact

SEQ ID NO: 52 - DNA, 20 bp, Artificial sequence
  Primer
ggccttggttctcaatgtgt

SEQ ID NO: 53 - DNA, 18 bp, Artificial sequence
  Primer
ctacttcgagggcgagca

SEQ ID NO: 54 - DNA, 20 bp, Artificial sequence
  Primer
tccagtggcttctgaccttc